(19) United States
(12) Patent Application Publication
(10) Pub. No.: US 2025/0134842 A1
(43) Pub. Date: May 1, 2025

(54) FATTY ACID MIMETICS AS MODULATORS OF NUCLEAR RECEPTOR TLX (NR2E1)

(71) Applicants: Trustees of Dartmouth College, Hanover, NH (US); University of Florida Research Foundation, Incorporated, Gainesville, FL (US)

(72) Inventors: Glenn C. Micalizio, Norwich, VT (US); Thomas P. Burris, Newberry, FL (US)

(21) Appl. No.: 18/930,348

(22) Filed: Oct. 29, 2024

Related U.S. Application Data
(60) Provisional application No. 63/595,155, filed on Nov. 1, 2023.

(51) Int. Cl. A61K 31/201; G01N 33/68

(52) U.S. Cl. CPC A61K 31/201; G01N 33/6875; G01N 2333/70567

(57) ABSTRACT

Fatty acid mimetics are identified as modulators of nuclear receptor TLX (NR2E1) in a subject in need thereof. Methods for treating a disease or condition associated with nuclear receptor TLX (NR2E1) comprise administering a fatty acid mimetic of Formula (I), Formula (II), Formula (III), or a pharmaceutically acceptable salt thereof. Methods for modulating activity of nuclear receptor TLX (NR2E1) or for promoting neurogenesis and/or neural stem cell proliferation include contacting the fatty acid mimetic with nuclear receptor TLX (NR2E1).

### CROSS REFERENCE TO RELATED APPLICATIONS

This patent application claims priority to U.S. Provisional Patent Application No. 63/595,155, filed on Nov. 1, 2023, the entire contents of which are fully incorporated herein by reference.

### FEDERALLY SPONSORED RESEARCH OR DEVELOPMENT

This invention was made with government support under R01 GM134725 awarded by the National Institutes of Health. The government has certain rights in the invention.

### FIELD OF THE INVENTION

The present disclosure relates to modulators of the nuclear receptor TLX (NR2E1) and methods of screening compounds for activity as modulators of the nuclear receptor TLX (NR2E1).

### BACKGROUND OF THE INVENTION

Nuclear receptors (NRs) are an important group of ligand-dependent transcriptional factors that regulate development, maintain homeostasis, and cause diseases and conditions. Nuclear receptors control or change gene expression in response to binding ligands such as steroids, lipophilic signaling molecules, and others. For a large group of NRs, natural ligands or even synthetic ligands have not been identified or confirmed, and such NRs are often referred to as orphan nuclear receptors.

The nuclear receptor “tailless” or TLX is also known as nuclear receptor subfamily 2, group E, member 1 (NR2E1). Nuclear receptor TLX (NR2E1) is a transcriptional repressor that is primarily expressed in the forebrain of vertebrates. Nuclear receptor TLX can engage in transcriptional regulation and act as a transcriptional repressor in some instances. Nuclear receptor TLX plays an important or essential role in embryonic and adult neurogenesis, in adult neural stem cell (NSC) proliferation, self-renewal, and differentiation, and in the pathogenesis of glioblastomas. Nuclear receptor TLX possesses a DNA binding domain, a hinge region, and a Ligand Binding Domain (LBD), and lacks an N-terminal domain containing the ligand-independent activation function. Griffett K et al., Cell Chem Biol 2020 Oct. 15; 27 (10): 1272-1284.e4, discusses natural and synthetic retinoids which were identified as functional ligands for TLX. The natural retinoid all-trans retinaldehyde (ATRAL) and synthetic retinoids were said to directly bind to TLX and either activate or inhibit its transcriptional repressor activity. Kandel P, et al., Proc Natl Acad Sci USA. 2022 Mar. 29; 119 (13): e2023784119, states that oleic acid is an endogenous ligand of nuclear receptor TLX (NR2E1) and serves as a metabolic regulator of TLX activity that can be used to selectively target neural stem cells, paving the way for future therapeutic manipulations to counteract pathogenic impairments of neurogenesis

Oleic acid is a conformationally heterogeneous fatty acid that has a regulatory effect at many different receptors. However, the structural basis for regulatory function by oleic acid at fatty acid receptors is difficult to determine. The structure of oleic acid is shown in FIG. 1.

Notably, the double bond present in oleic acid is the only moiety present that offers a conformational constraint for this molecule. Specifically, the stereochemistry of this functional group (the alkene) controls where the relative position of the two sp3 carbons bound to it will reside in 3-D space in relation to the plane of the double bond. Moving beyond those two sp3 carbons that are attached to the alkene, the vast majority of the hydrocarbon chains stemming from the central alkene are conformationally unorganized. As such, in the absence of structural data that reveals how oleic acid binds to a receptor of interest, it is not possible to accurately predict the conformation that oleic acid takes when bound.

There remains a need for greater understanding of natural and synthetic ligand binding to nuclear receptor TLX (NR2E1), including the conformation of ligands when bound. There is also a need for synthetic ligands that can modulate activity of nuclear receptor TLX (NR2E1).

### SUMMARY OF THE INVENTION

The present disclosure relates to novel methods for treating a disease or condition associated with nuclear receptor TLX (NR2E1) in a subject in need thereof. The methods comprise administering to the subject a therapeutically effective amount of a compound of Formula (I), Formula (II), Formula (III), or a pharmaceutically acceptable salt thereof. Formulas (I), (II) and (III) are shown and described in detail below.

The present disclosure also relates to novel methods for modulating activity of nuclear receptor TLX (NR2E1). The methods comprise contacting nuclear receptor TLX with a compound of Formula (I), Formula (II), Formula (III), or a pharmaceutically acceptable salt thereof. In some embodiments, the compound is an agonist of nuclear receptor TLX (NR2E1). In some embodiments, the compound is an antagonist or inverse agonist of nuclear receptor TLX (NR2E1).

The present disclosure also relates to novel methods for promoting neurogenesis and/or neural stem cell proliferation. The method comprise contacting a cell with a compound of Formula (I), Formula (II), Formula (III), or a pharmaceutically acceptable salt thereof. In some embodiments, the compound is a modulator (e.g., an agonist) of nuclear receptor TLX (NR2E1).

In some embodiments of the present methods, the compound or salt thereof is a fatty acid mimetic. In certain embodiments, the fatty acid mimetic is a chiral and conformationally constrained enantioenriched variant of a natural fatty acid.

### BRIEF DESCRIPTION OF THE DRAWINGS

FIG. 1 depicts the structure of oleic acid.

FIG. 2 depicts a schematic structure of exemplary oleic acid mimetics.

FIG. 3 depicts a collection of motifs that offer local conformational bias.

FIG. 4 depicts selected motifs to achieve rigidification.

FIG. 5 depicts four “classes” of mimetics that differ by the position of the conformational constraint.

FIG. 6 depicts chemistry capable of systematically making a fatty acid mimetic.

FIG. 7 depicts traces of a carbon backbone through a diamond lattice to provide examples of different conformations.

FIG. 8A depicts the structure of the alkynes and allylic alcohols employed.

FIG. 8B depicts examples of the chemical pathway for the synthesis of members of the fatty acid mimetic collection. Yield includes all isomers after deprotection: 21-rs=3:1, Z:E (of major regioisomer)=13:1; 22-rs=4:1, Z:E (of major regioisomer)=10:1; 23-rs=4:1, Z:E (of major regioisomer)=7:1, only one alkene stereoisomer visible via ¹HNMR for the minor regioisomeric coupling product.

FIG. 9A to 9Z show the measurements for oleic acid, linoleic acid, and Compounds 1-A to 4-F in the AlphaScreen assay.

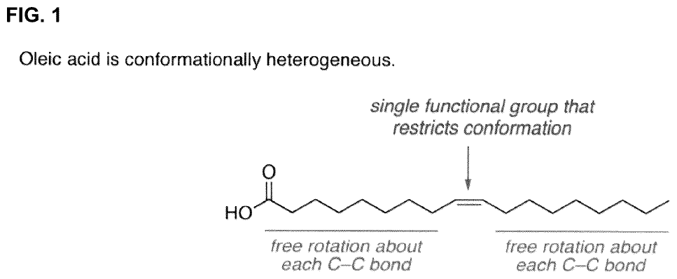

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

Throughout the present description, whenever a general method of using a compound or salt thereof is discussed, it should be understood that the discussion relates to and encompasses any specific methods of use discussed herein or elsewhere, including later-developed methods of use within the scope of the present teachings, By way of example, the general methods of using a compound or salt described herein include, but are not limited to, specific methods of use such as methods for treating a disease or condition associated with nuclear receptor TLX (NR2E1) in a subject in need thereof, methods for modulating activity of nuclear receptor TLX (NR2E1), methods for promoting neurogenesis and/or neural stem cell proliferation, and other specific methods of use that will become apparent to skilled persons from the present teachings.

In one aspect, the present disclosure relates to methods of using a fatty acid mimetic comprising a conformationally constrained core comprising two or more conformational constraints and at least one optional flexible subunit. In some such embodiments, the flexible subunit (e.g., an alkylene, such as a methylene) is interspersed between the two conformational constraints. In certain embodiments, the hydrocarbon length of oleic acid (18) is a common characteristic of the mimetic.

In one aspect, the present disclosure relates to methods of using a compound of Formula (I) or a pharmaceutically acceptable salt thereof, wherein A, X, R, m and n are as defined below.

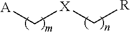

In some embodiments, the compound of Formula (I) or salt thereof is used for treating a disease or condition associated with nuclear receptor TLX (NR2E1) in a subject in need thereof. In some embodiments, the compound of Formula (I) or salt thereof is used for modulating activity of nuclear receptor TLX (NR2E1). In some embodiments, the compound of Formula (I) or salt thereof is used for promoting neurogenesis and/or neural stem cell proliferation.

In one aspect, the present disclosure relates to methods of using a compound of Formula (II) or a pharmaceutically acceptable salt thereof, wherein X, R¹, m and n are as defined below.

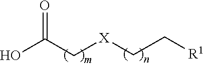

In some embodiments, the compound of Formula (II) or salt thereof is used for treating a disease or condition associated with nuclear receptor TLX (NR2E1) in a subject in need thereof. In some embodiments, the compound of Formula (II) or salt thereof is used for modulating activity of nuclear receptor TLX (NR2E1). In some embodiments, the compound of Formula (II) or salt thereof is used for promoting neurogenesis and/or neural stem cell proliferation.

In one aspect, the present disclosure relates to methods of using a compound of Formula (III) or a pharmaceutically acceptable salt thereof, wherein X¹, X², m and n are as defined below.

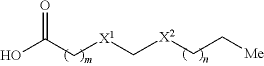

In some embodiments, the compound of Formula (III) or salt thereof is used for treating a disease or condition associated with nuclear receptor TLX (NR2E1) in a subject in need thereof. In some embodiments, the compound of Formula (III) or salt thereof is used for modulating activity of nuclear receptor TLX (NR2E1). In some embodiments, the compound of Formula (III) or salt thereof is used for promoting neurogenesis and/or neural stem cell proliferation.

### A. Definitions

As used in the specification and the appended claims, unless specified to the contrary, the following terms have the meaning indicated:

As used herein, the terms “a,” “an,” and “the” include both singular and plural referents, unless the context clearly dictates otherwise. Thus, for example, “a compound” includes one compound and plural compounds. The terms “first” and “second” are terms to distinguish different elements, not terms supplying a numerical limit, and a device having first and second element can also include a third, a fourth, a fifth, and so on, unless otherwise indicated.

The term “modulator” refers to a substance (e.g., a ligand) that binds to and initiates, stimulates, inhibits, arrests, regulates, or otherwise changes the activity of a receptor. The term “modulate” and “modulating” as used herein refer to the effect(s) of such modulators from binding to a receptor. Modulators can act on different parts of receptors and regulate activity in a positive, negative, or neutral direction with varying degrees of efficacy. Modulators include agonists and antagonists, as well as partial agonists, inverse agonists, orthosteric modulators, and allosteric modulators. An “agonist” is a substance that increases functional activity of a receptor (i.e., signal transduction through the receptor). An “antagonist” is a substance that decreases functional activity of a receptor either by inhibiting the action of an agonist or by its own activity (inverse agonist). A “selective modulator” is a substance that modulates the activity of a particular combination of a nuclear receptor and one or more polypeptides from a group of coactivators, corepressor, kinase or signaling molecule.

As disclosed herein, numeric ranges are provided for various parameters or data. It should be understood that numeric ranges also disclose include each intervening value within the range, to the tenth of the unit of the lower limit, unless the context clearly dictates otherwise. It should also be understood that the upper and lower limits may each be included or excluded in a range.

It should be recognized that chemical structures and formula may be elongated or enlarged for illustrative purposes.

The term “about” as used herein means approximately, and in most cases within 10% of the stated value.

The term “substituted” in reference to alkyl, heteroalkyl, alkenyl, heteroalkenyl, alkynyl, heteroalkynyl, aryl, heteroaryl, heterocycle etc., for example, “substituted alkyl”, “substituted heteroalkyl”, “substituted alkenyl”, substituted heteroalkenyl”, “substituted alkynyl”, “substituted heteroalkynyl”, “substituted aryl”, “substituted heteroaryl”, and “substituted heterocycle” means alkyl, heteroalkyl, alkenyl, heteroalkenyl, alkynyl, heteroalkynyl, aryl, heteroaryl, heterocycle, respectively, in which one or more hydrogen atoms are each independently replaced with a non-hydrogen substituent or with a deuterium atom. Typical substituents include, but are not limited to, —R¹², —R¹⁴, —O—, ═O, —OR¹⁴, —SR¹⁴, —S—, —NR¹⁴₂, —N⁺R¹⁴₃, ═NR¹⁴, —C(R¹²)₃, —CN, —OCN, —SCN, —N═C═O, —NCS, —NO, —NO₂, ═N₂, —N₃, —NHC(═O)R¹⁴, —NHS(═O)₂R¹⁴, —C(═O)R¹⁴, —C(═O)N(R¹⁴)₂—S(═O)₂O—, —S(═O)₂OH, —S(═O)₂R¹⁴, —OS(═O)₂OR¹⁴, —S(═O)₂NR¹⁴, —S(═O)R¹⁴, —OP(═O)(OR¹⁴)₂, —P(═O)(OR¹⁴)₂, —P(═O)(O—)₂, —P(═O)(OH)₂, —P(O)(OR¹⁴)(O—), —C(═O)R¹⁴, —C(═O)OR¹⁴, —C(═O)R¹², —C(S)R¹⁴, —C(O)OR¹⁴, —C(O)O—, —C(S)OR¹⁴, —C(O)SR¹⁴, —C(S)SR¹⁴, —C(O)N(R¹⁴)₂, —C(S)N(R¹⁴)₂, —C(═NR¹⁴)N(R¹⁴)₂, where each R¹² is independently a halogen: F, Cl, Br, or I; and each R¹⁴ is independently hydrogen, deuterium, alkyl, aryl, arylalkyl, a heterocycle, or a protecting group. When the number of carbon atoms is designated for a substituted group, the number of carbon atoms refers to the group, not the substituent (unless otherwise indicated). For example, a C₁₋₄ substituted alkyl refers to a C₁₋₄ alkyl, which can be substituted with groups having more than, e.g., 4 carbon atoms.

The terms “alkyl” and “alkylene” refer to a straight-chain or branched alkyl, preferably having 1, 2, 3, 4, 5, 6, 7, 8, 9, 10, 11, 12, 13, 14, 15, 16, 17, 18, 19, 20, 21, 22, 23 or 24 carbons. Examples of such alkyl groups include methyl, ethyl, propyl, isopropyl, n-butyl, sec-butyl, isobutyl, tert-butyl, pentyl, isoamyl, hexyl, and the like. Alkyl groups may be unsubstituted or substituted, as defined above.

The term “alkenyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon double bonds. Nonlimiting examples of alkenyl groups include ethenyl, 1-propenyl, 2-propenyl (allyl), iso-propenyl, 2-methyl-1-propenyl, 1-butenyl, and 2-butenyl. Alkenyl groups may be unsubstituted or substituted by one or more suitable substituents, as defined above.

The term “alkynyl” refers to a straight or branched hydrocarbon, preferably having 2, 3, 4, 5, 6, 7, 8, 9, 10, 11, 12, 13, 14, 15, 16, 17, 18, 19, 20, 21, 22, 23 or 24 carbons, and having one or more carbon-carbon triple bonds. Alkynyl groups include, but are not limited to, ethynyl, propynyl, and butynyl. Alkynyl groups may be unsubstituted or substituted by one or more suitable substituents, as defined above.

Whenever a range of the number of atoms in a structure is indicated (e.g., a C₁-C₂₄ alkyl, C₂-C₂₄ alkenyl, C₂-C₂₄ alkynyl, etc.), it is specifically contemplated that the structure can have any individual number of carbon atoms falling within the indicated range. By way of example, a description of the group such as an alkyl group using the recitation of a range of 1-25 carbon atoms (e.g., C₁-C₂₅) encompasses and specifically describes an alkyl group having any of 1, 2, 3, 4, 5, 6, 7, 8, 9, 10, 11, 12, 13, 14, 15, 16, 17, 18, 19, 20, 21, 22, 23, 24 and 25 carbon atoms. It is also contemplated that any sub-range can be formed from such a range. As examples, a reference to a C₁-C₂₅ alkyl, or a reference to m or n being 1 to 25 in Formulas (I, II, and III), should be understood as disclosing the sub-ranges of 1-7 carbon atoms (e.g., C₁-C₇), 1-6 carbon atoms (e.g., C₁-C₆), 1-4 carbon atoms (e.g., C₁-C₄), 1-3 carbon atoms (e.g., C₁-C₃), or 2-24 carbon atoms (e.g., C₂-C₂₄) as well.

“Heteroalkyl”, “heteroalkenyl” and “heteroalkynyl” refers respectively to a molecule comprising an alkyl group, an alkenyl group and an alkynyl group, in which one or more carbon atoms have been replaced with a heteroatom, such as, O, N, or S, except that the carbon atom of the alkene or alkyne are not replaced. Other carbons within the molecule, including carbons of an alkyl group, can be replaced independently with a heteroatom (O, N, or S), meaning the first carbon, the terminal carbon or an internal carbon. A “substituted heteroalkyl”, a “substituted heteroalkenyl”, or a “substituted heteroalkynyl” refers to a molecule comprising a heteroalkyl, a heteroalkenyl or a heteroalkynyl group as defined herein, and one or more hydrogen atom has been replaced with a substituent (as defined above in the “substituted” definition).

The term “aryl” refers to an unsubstituted or substituted aromatic carbocyclic substituent, such as phenyl, naphthyl, anthracyl, indanyl, and the like. It is understood that the term aryl applies to cyclic substituents that are planar and comprise 4n+2 electrons, according to Hückel's Rule. Aryl groups may be unsubstituted or substituted by one or more suitable substituents, as defined above, and include variants that are polycyclic wherein at least one of rings is aromatic. The term “heteroaryl” refers to a monocyclic or bicyclic 5- or 6-membered ring system, wherein the heteroaryl group is unsaturated and satisfies Hückel's rule.

The term “carbonyl” refers to a substituent comprising a carbon double bonded to an oxygen. Examples of such substituents include aldehydes, ketones, carboxylic acids, esters, amides, carbonates, and carbamates. Carbonyl groups may be unsubstituted or substituted by one or more suitable substituents, as defined above.

The term “amino” refers to any nitrogen-containing moiety. Non-limiting examples of the amino group are NH₂— (primary), R¹⁵HN— (secondary), and (R¹⁵)₂N— (tertiary) where R¹⁵ is alkyl, alkenyl, alkynyl, aryl, heterocyclic, or heteroaryl.

The term “deuterated” refers to one or more hydrogen atoms being replaced by deuterium. For example, a deuterated alkyl is an alkyl wherein one or more hydrogen atoms in the alkyl group are replaced by deuterium. A deuterated group can be fully deuterated, wherein all hydrogens in the group are replaced by deuterium. Alternatively a deuterated group can be partially deuterated, wherein fewer than all hydrogens are replaced by deuterium. In the present disclosure, deuterium may be denoted by d or D, and a methyl group where all the three hydrogen atoms are replaced by deuterium atoms may be denoted by methyl-d3, d3-methyl, fully deuterated methyl, or CD₃.

The term “pharmaceutically acceptable” is used adjectivally to mean that the modified noun is appropriate for use as a pharmaceutical product for human use or as a part of a pharmaceutical product for human use.

“Halogen” or “halo” refers to fluorine, chlorine, bromine, and iodine.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference.

### B. Methods of Using Fatty Acid Mimetics

In one aspect, the disclosure provides methods of using a compound (e.g., a fatty acid mimetic) of Formula (I):

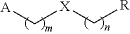

or a pharmaceutically acceptable salt thereof; wherein A comprises a polar head group; X comprises a conformationally constrained core comprising two or more conformational constraints and at least one optional flexible subunit; R comprises a non-polar group, generally intended to mimic the non-polar tail of an endogenous fatty acid ligand; and each of m and n is independently an integer from 1 to 25, or from 1 to 19, or from 1 to 11, or from 1 to 7.

In some embodiments, the compound of Formula (I) or salt thereof is used for treating a disease or condition associated with nuclear receptor TLX (NR2E1) in a subject in need thereof. In some embodiments, the compound of Formula (I) or salt thereof is used for modulating activity of nuclear receptor TLX (NR2E1). In some embodiments, the compound of Formula (I) or salt thereof is used for promoting neurogenesis and/or neural stem cell proliferation. In certain embodiments, the flexible subunit is an alkylene. In some such embodiments, the flexible subunit is a methylene subunit.

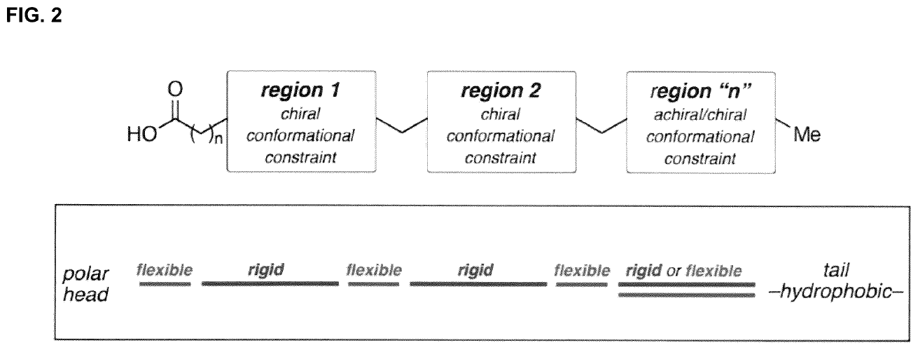

The present disclosure describes uses of fatty acid (e.g., oleic acid) mimetics having at least one and, preferably, at least two chiral conformational constraints. In certain embodiments, the fatty acid mimetic comprises a polar head, a first chiral conformational constraint (labeled as “region 1” in FIG. 2), a second chiral conformational constraint (“region 2”), a flexible subunit interspersed between the two conformational constraints, and a hydrophobic tail. A schematic structure of exemplary oleic acid mimetics is shown in FIG. 2.

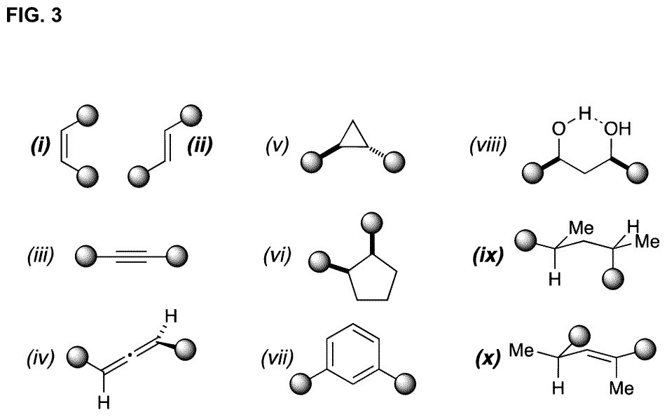

The conformational constraints are inspired by the structural motifs shown in FIG. 3. The conformational constraints depicted in FIG. 3 can be characterized as acyclic conformational constraints (e.g., (i), (ii), (iii), (iv), (ix), and/or (x)); simple π-unsaturation conformational constraints (e.g., (i)-(iv)); acyclic chiral conformational constraints (e.g., (ix) and/or (x)); and cyclic conformational constraints (e.g., (v)-(viii) where it is understood that saturated and unsaturated variants would be useful). The circles indicate the conformational constraint's points of attachment to other portions of the compound.

In some embodiments, the compound used in the present methods has a structure of Formula (I-A):

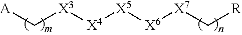

wherein
X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.

In some embodiments, at least one of X³ or X⁷ is a conformational constraint, such as one of the conformational constraints (i) to (x) in FIG. 3 above. In some embodiments, at least one of X³ or X⁷ (or both of X³ or X⁷) is an acyclic conformational constraint (e.g., (i), (ii), (iii), (iv), (ix), and/or (x) about); or a simple π-unsaturated conformational constraint (e.g., (i)-(iv)); or an acyclic chiral conformational constraints (e.g., (ix) and/or (x)); or a cyclic conformational constraint (e.g., (v)-(viii)). In some embodiments, at least one of X³ or X⁷ (or both of X³ and X⁷) is a conformational constraint comprising a ring system. In some embodiments, at least one of X³ or X⁷ (or both of X³ and X⁷) is a cycloalkyl, such as (For example) 1,2-cis-disubstituted cyclopentane, 1,2-trans-disubstituted cyclopentane, cis-substituted cyclopropanes, trans-substituted cyclopropanes, or 1,3-cis-disubstituted tetrahydrofuran. In some embodiments, X⁴ and X⁶ are independently C₁₋₆-alkylene, such as a C₁₋₃-alkylene (e.g., methylene, ethylene, or propylene).

A comprises a polar head group. In some embodiments, A consists of a polar head group. In some embodiments, A comprises A²-R¹⁵, wherein A² is a polar head group, and R¹⁵ is a bond, a C₁₋₆-alkyl, or a C₂₋₆-alkenyl. In some embodiments, A or A² comprises a carboxylic acid, a carboxyl ester, an amide, a ketone, a phosphonic acid, a phosphinic acid, a sulfonic acid, a sulfinic acid, a sulfonamide, an acyl-sulfonamide, a hydroxamic acid, a hydroxamic ester, a sulfonylurea, an acylurea, a tetrazole, a thiazolidinedione, an oxazolidinedione, an oxadiazolone, a thiadiazolone, an oxathiadiazole oxide, an oxadiazolethione, an isoxazole, a tetramic acid, a cyclopentane diones, a phenol derivative, a squaric acid derivative, or a salt thereof. Exemplary polar head groups include, but are not limited to, those in Table 1 (wherein the circle indicates the groups point of attachment to the rest of the compound of Formula (I or I-A):

TABLE 1

| | |
|---|---|
| Carboxylic acids | 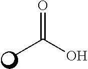 |
| Carboxyl esters | 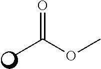 |
| Amides | 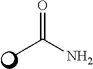 |
| Ketones | 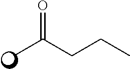 |
| Hydroxamic acids | 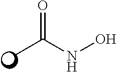 |
| | 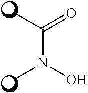 |
| Hydroxamic esters | 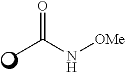 |
| | 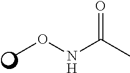 |
| Phosphonic acid | 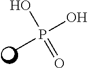 |
| Phosphinic acid | 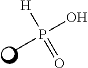 |
| Sulfonic acid (sodium salt) | 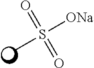 |
| Sulfinic acid (sodium salt) | 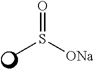 |
| Sulfonamides | 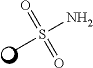 |
| | 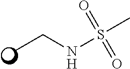 |
| Acyl-sulfonamides | 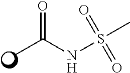 |
| | 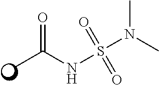 |
| Sulfonylurea | 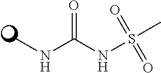 |
| Acylurea | 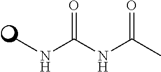 |
| Tetrazole | 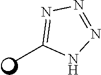 |
| Thiazolidine dione | 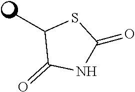 |
| Oxazolidine dione | 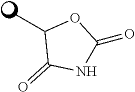 |
| Oxadiazol-5(4H)-one | 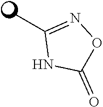 |
| Thiadiazol-5(4H)-one | 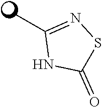 |
| Oxathiadiazole-2-oxide | 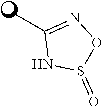 |
| Oxadiazol-5(4H)-thione | 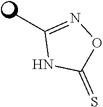 |
| Isoxazole | 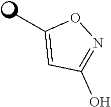 |
| Tetramic acid | 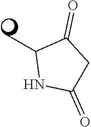 |
| Cyclopentane 1,3-diones | 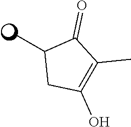 |
| | 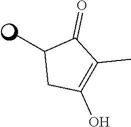 |
| | 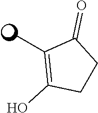 |
| Cyclopentane 1,2-diones | 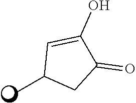 |
| Squaric acid derivatives | 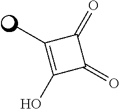 |
| | 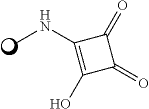 |
| Substituted phenols | 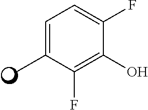 |
| | 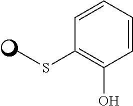 |
| | 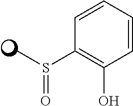 |
| | 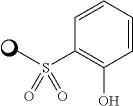 |

In one aspect, the disclosure provides methods of using a compound (e.g., a fatty acid mimetic) of Formula (II):

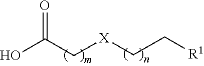

or a pharmaceutically acceptable salt thereof; wherein:
X comprises a constrained core comprising one or more acyclic conformational constraints and at least one optional flexible subunit;
R¹ is a C₁₋₆-alkyl; and
each of m and n is independently an integer from 1 to 25, or from 1 to 19, or from 1 to 11, or from 1 to 7;
provided that the compound is not a natural product and/or
provided that at least one conformational constraint is a conformational constraint other than

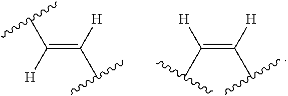

In some embodiments, the compound of Formula (II) or salt thereof is used for treating a disease or condition associated with nuclear receptor TLX (NR2E1) in a subject in need thereof. In some embodiments, the compound of Formula (II) or salt thereof is used for modulating activity of nuclear receptor TLX (NR2E1). In some embodiments, the compound of Formula (II) or salt thereof is used for promoting neurogenesis and/or neural stem cell proliferation.

In certain embodiments, the constrained core comprises (ix):

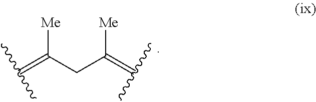 [SMILES: *=C(C)CC(=*)C]

In certain embodiments, the acyclic conformational constraints are independently are selected from the group consisting of (x), (x′), (x″), and (x″′):

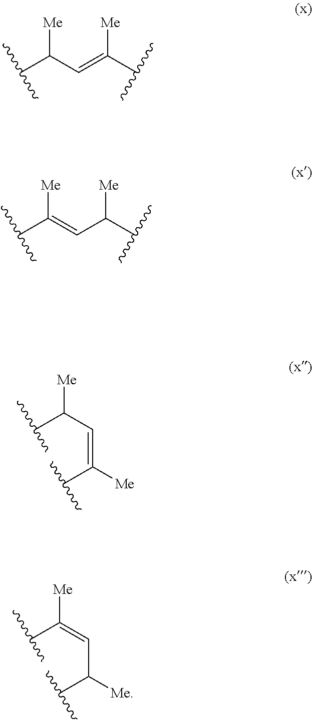 [SMILES: */C(C)=C/C(*)C ; */C(C)=C/C(*)C ; */C(C)=C\C(*)C ; */C(C)=C\C(*)C]

In certain embodiments, two or more conformational constraints are joined by a C₁₋₆-alkylene, such as a C₁₋₃-alkylene (e.g., methylene, ethylene, or propylene).

In one aspect, the disclosure provides a compound (e.g., a fatty acid mimetic) of Formula (III):

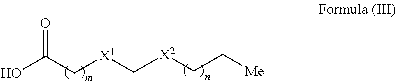

or a pharmaceutically acceptable salt thereof; wherein:
X¹ is

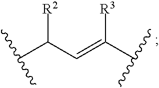

R² is C₁₋₆-alkyl;
R³ is C₁₋₆-alkyl;
X² is

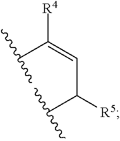

R⁴ is C₁₋₆-alkyl;
R⁵ is hydrogen or C₁₋₆-alkyl; and
each of m and n is independently an integer from 1 to 25, or from 1 to 19, or from 1 to 11, or from 1 to 7.

In some embodiments, the compound of Formula (III) or salt thereof is used for treating a disease or condition associated with nuclear receptor TLX (NR2E1) in a subject in need thereof. In some embodiments, the compound of Formula (III) or salt thereof is used for modulating activity of nuclear receptor TLX (NR2E1). In some embodiments, the compound of Formula (III) or salt thereof is used for promoting neurogenesis and/or neural stem cell proliferation.

In Formulas (I), (II) and (Ill), each of m and n can independently be 1, 2, 3, 4, 5, 6, 7, 8, 9, 10, 11, 12, 13, 14, 15, 16, 17, 18, 19, 20, 21, 22, 23, 24, or 25. In certain embodiments, m is 1, 3, 5, 7, 9, 11, 13, 15, 17, 19, 21, 23 or 25. In certain embodiments of the Formulas, n is 1, 3, 5, 7, 9, 11, 13, 15, 17, 19, 21, 23 or 25. In certain embodiments, m+n is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In certain embodiments, X¹ is (x.1) or (x.2):

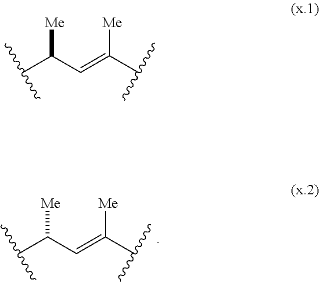 [SMILES: */C(C)=C/[C@@H](*)C ; */C(C)=C/[C@H](*)C]

In certain embodiments, X² is (x″′.1) or (x″′.2):

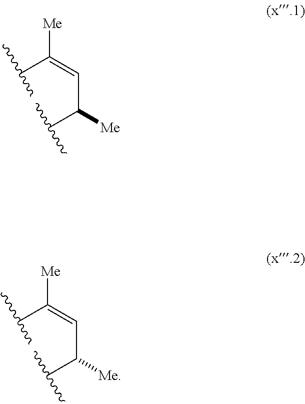 [SMILES: */C(C)=C\[C@H](*)C ; */C(C)=C\[C@@H](*)C]

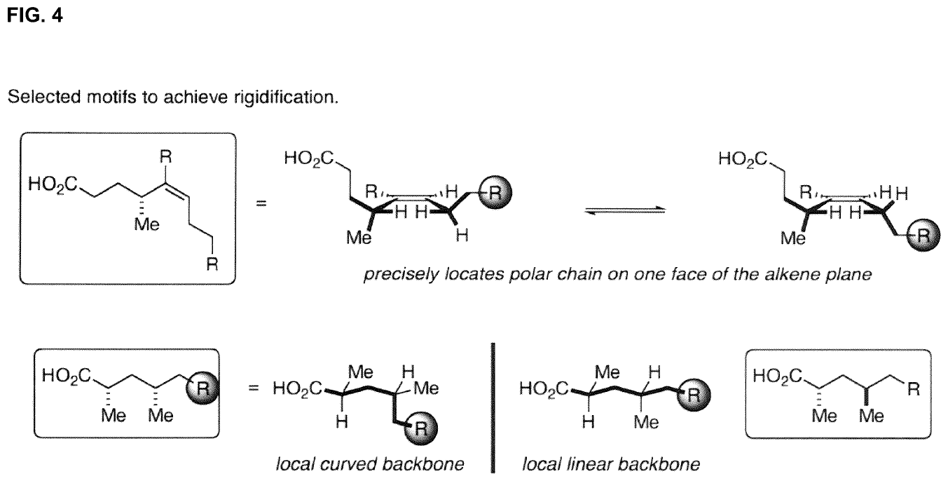

In certain embodiments for any aspect of the invention, the nature of the chiral conformational constraint is based on a structural motif observed in polyketide natural product such as those shown in FIG. 4 (avoiding allylic strain and eclipsing 1,5-interactions (aka, syn-pentane interactions)).

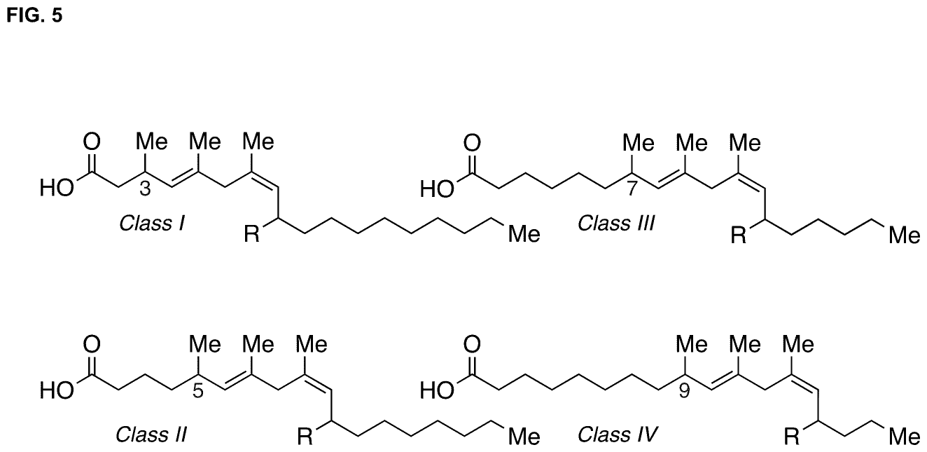

Exemplary compounds include Class I, II, III, IV compounds shown in FIG. 5 where “R” is a hydrogen or C₁₋₆-alkyl. Notably, the conformational constraints within each of these “classes” are positioned at different locations within the fatty acid carbon backbone (for “Class I”, the constraint begins at C3, for “Class II”, the constraint begins at C5, for “Class III”, the constraint begins at C7, and for “Class IV”, the constraint begins at C9).

In one aspect, compounds disclosed herein possess biological activity, for example, by modulating TLX.

### C. Methods of Making Fatty Acid Mimetics

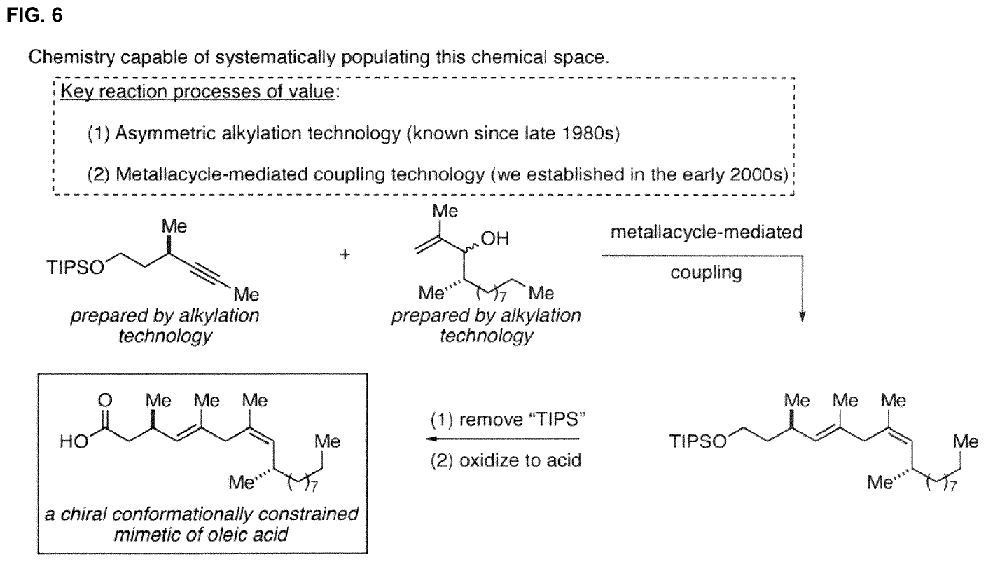

The compound (e.g., a fatty acid mimetic) of any of Formulas (I), (II) or (III) can be made by the methods disclosed herein or other suitable methods that may be apparent to skilled persons based on the teachings of the present description. FIG. 6 summarizes a suitable method of making the compounds. As shown in FIG. 6 the chemistry to make the compounds leverages asymmetric alkylation chemistry well known to those skilled in the art, and includes a modern metallacycle-mediated coupling reaction between an alkyne and an allylic alcohol. The synthetic pathway is modular/convergent and allowed for synthesis of fatty acid mimetics that contain the type of conformational constraints depicted in FIG. 6 at different positions within a fatty acid mimetic (in the case depicted, the compounds were designed to contain a C₁₈ backbone, inspired by oleic acid).

Exemplary synthetic methods, including a retrosynthetic strategy for compounds disclosed herein, are disclosed in Markham et al., ACS Cent. Sci., 10, 477-486, 20024 and WO2024/211164 A2, the entire contents of which are fully incorporated herein by reference.

In some embodiments, the method of making the compound comprises coupling of an alkyne and an allylic alcohol, such as by metallacycle-mediated coupling. One skilled in the art may contemplate alternative methods for the construction of such targets in light of the teachings of the present disclosure, including the use of Pd-catalyzed cross-coupling technology (albeit with the understanding the nature of the coupling partners would have to be compatible with such transition metal catalyzed coupling chemistry).

In some embodiments, a method is provided for making a compound of Formula (I), wherein the method comprises coupling an alkyne of Formula (IV):

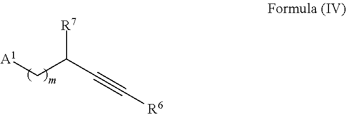

wherein A¹ comprises a precursor to a polar head group (like a protected alcohol) or the actual targeted polar head group; R⁶ is a C₁₋₆-alkyl; R⁷ is hydrogen or a C₁₋₆-alkyl; with an allylic alcohol of Formula (V):

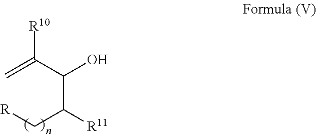

wherein R comprises a group intended to mimic the non-polar tail of endogenous fatty acids; R¹⁰ is C₁₋₆-alkyl; R¹¹ is hydrogen or a C₁₋₆-alkyl; and each of m and n is independently an integer from 1 to 25, or from 1 to 19, or from 1 to 11, or from 1 to 7. The coupling can be a metallacycle-mediated coupling, which can be performed in the presence of a titanium alkoxide, such as Ti(Oi-Pr)₄, and an organometallic reductant [such as n-butyl lithium (n-BuLi) or i-PrMgX)], and can be conducted in the presence of a silylating agent, such as trimethylsilyl chloride (TMSCl).

Additional information regarding metallacycle-mediated coupling and synthesis of 1,4-dienes in a convergent fashion can be found in (a) Kolundzic, F.; Micalizio, G. C.; Synthesis of Substituted 1,4-Dienes by Direct Alkylation of Allylic Alcohols. J. Am. Chem. Soc. 2007, 129, 15112. (b) Diez, P. S.; Micalizio G. C. Chemoselective Reductive Cross-Coupling of 1,5-Diene-3-ols with Alkynes: A Facile Entry to Stereodefined Skipped Trienes. J. Am. Chem. Soc. 2010, 132, 9576-9578. (c) Jeso, V.; Micalizio G. C. Total Synthesis of Lehualide B by Allylic Alcohol-Alkyne Reductive Cross-Coupling. J. Am. Chem. Soc. 2010, 132, 11422-11424. (d) Diez, P. S.; Micalizio, G. C. Convergent Synthesis of Deoxypropionates. Angew. Chem. Int. Ed. Engl. 2012, 51, 5152-5156.

Exemplary silylating agents include, but are not limited to trimethylsilyl chloride (TMSCl), trimethylsilyl bromide (TMSBr), and chlorotriethylsilane (TESCl).

In some embodiments, the alkyne has a structure of Formula (IV-A):

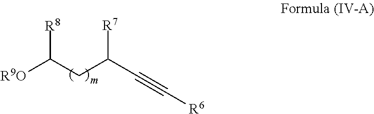

wherein R⁸ is hydrogen or a C₁₋₆-alkyl; and R⁹ is hydrogen or an oxygen protecting group. In some embodiments, the compound has a structure of Formula (IV-A-1 or (IV-A-2):

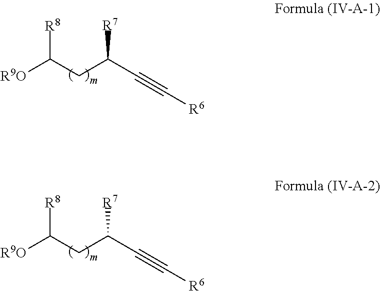

wherein R⁷ is a C₁₋₆-alkyl. In some embodiments of Formulas (IV-A, IV-A-1, or IV-A-2), R⁸ is hydrogen; and R⁹ is an oxygen protecting group. In some embodiments, R⁹ is an oxygen protecting group selected from methyl, tert-butyloxycarbonyl (BOC), methoxymethyl (MOM), tert-butyldimethylsilyl (TBS), tert-butyldiphenylsilyl (TBDPS), tribenzylsilyl, and triisopropylsilyl (TIPS).

Examples of oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), t-butoxymethyl, 4-pentenyloxymethyl (POM), 2-methoxyethoxymethyl (MEM), tetrahydropyranyl (THP), 2-trimethylsilylethyl, t-butyl, p-chlorophenyl, p-methoxyphenyl, benzyl (Bn), p-methoxybenzyl, 3,4-dimethoxybenzyl, p-halobenzyl, 2,6-dichlorobenzyl, p-phenylbenzyl, diphenylmethyl, 5-dibenzosuberyl, triphenylmethyl, trimethylsilyl (TMS), triethylsilyl (TES), triisopropylsilyl (TIPS), dimethylisopropylsilyl (IPDMS), diethylisopropylsilyl (DEIPS), t-butyldimethylsilyl (TBDMS), t-butyldiphenylsilyl (TBDPS), tribenzylsilyl, triphenylsilyl, diphenylmethylsilyl (DPMS), 2-methoxyethoxymethyl (MEM), 2-trimethylsilylethoxymethyl (SEM), methylthiomethyl (MTM), methoxytrityl (MMT), and dimethoxytrityl (DMT).

In some embodiments, the present methods further comprise making the alkyne by carbonyl alkynylation of a compound of Formula (VI or VI-A):

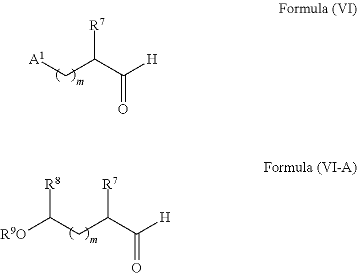

In some embodiments, the allylic alcohol has a structure of Formula (V-A or V-A-1 or V-A-2):

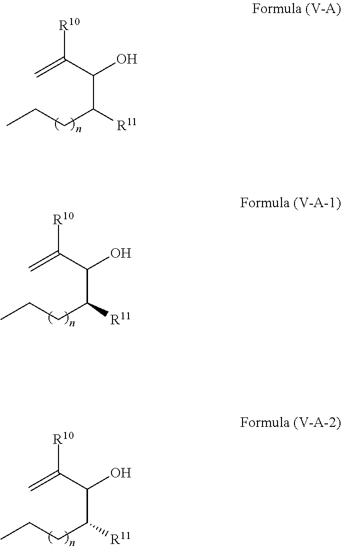

wherein R¹¹ is a C₁₋₆-alkyl. The stereochemistry at the allylic position is generally not critical and can be selected by those practicing the method. In some embodiments, the allylic position is a mixture of stereoisomers. In some embodiments, the allylic alcohol is enriched at the allylic position as mostly (S) or (R).

In some embodiments, the method further comprises making the allylic alcohol of Formula (V or V-A) by reaction of a compound of Formula (VII or VII-A) to convert aldehyde to allylic alcohol in a targeted manner:

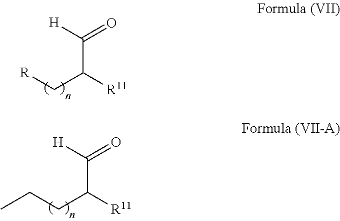

There are many available methods for the addition of vinyl groups to aldehydes, including other vinyl organometallic reagents used stoichiometrically or generated catalytically (e.g., NHK-coupling). Accordingly, the method can comprise making the allylic alcohol of Formula (V or V-A) by a reaction which comprises (a) a reaction with a Grignard reagent, e.g., (R¹⁰)(CH₂)C—MgR¹², where R¹² is a halogen; (b) a reaction with a vinyl lithium reagent; (c) catalytic addition by NHK coupling, or (d) reaction of an organolanthanum or organocerium reagent to an aldehyde.

In some embodiments, the present methods further comprise making the compound of any of Formulas (VI, VI-A, VII, or VII-A) by stereoselective alkylation of a compound of Formula (VIII) by methods known to those skilled in the art:

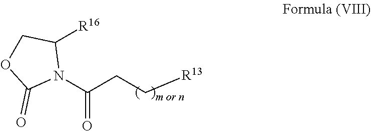

wherein R¹³ is C₁₋₆-alkyl or A¹; and R¹⁶ is a hydrogen, alkyl, aryl or arylalkyl.

In some embodiments, the present methods comprise stereoselectively alkylating a compound of Formula (VIII) wherein R¹³ is C₁₋₆-alkyl. After removal of the auxiliary and altering the oxidation state of the carbon previously attached to nitrogen by methods well known in the art, one can arrive at a compound of Formula (VII):

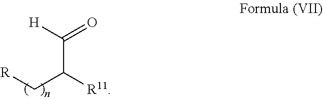

The compound of Formula (VII) is reacted with (R¹⁰)(CH₂)C—MgR¹² in a Grignard reaction to form the allylic alcohol of Formula (V). The methods also comprise stereoselectively alkylating a compound of Formula (VIII) wherein R¹³ is A¹, to form a compound of Formula (VI):

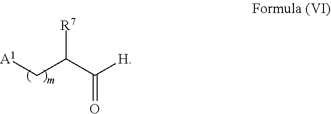

The methods also comprise making the alkyne of Formula (IV) by carbonyl alkynylation of the compound of Formula (VI). The present methods can then comprise coupling the alkyne of Formula (IV) and the allylic alcohol of Formula (V) as described above.

In some embodiments, A¹ comprises a precursor to the desired polar head group suitably protected with a protecting group (e.g., triisopropylsilyl), and the method further comprises deprotecting and oxidizing the head group to form a carboxylic acid group.

### D. Methods of Using Fatty Acid Mimetics

The present disclosure provides several novel methods of using the compounds of Formulas (I), (II) or (Ill), or pharmaceutically acceptable salts thereof. By way of example, methods of using a compound or salt described herein include, but are not limited to, methods such as treating a disease or condition associated with nuclear receptor TLX (NR2E1) in a subject in need thereof; modulating activity of nuclear receptor TLX (NR2E1); promoting neurogenesis and/or neural stem cell proliferation, and other specific uses that will become apparent to skilled persons from the present teachings.

In one aspect, the methods are for treatment of a disease or condition associated with nuclear receptor TLX (NR2E1) in a subject in need thereof. Diseases and conditions associated with nuclear receptor TLX (NR2E1) include, but are not limited to, neurological disorders; retinopathies; cancers; and metabolic disorders. The methods include a step of administering to the subject a compound according to Formulas (I), (II) or (Ill), or a pharmaceutically acceptable salt thereof. The subject can be an animal such as a mammal, including, but not limited to, primates (e.g., humans), cows, sheep, goats, horses, dogs, cats, rabbits, rats, mice and the like. Preferably the subject is a human.

In some embodiments, the neurological disorder is cognitive dysfunction, mild cognitive impairment, depression, major depressive disorder (MDD), schizophrenia, Alzheimer's disease, Parkinson's disease, early onset dementia, multiple sclerosis, irradiation-induced cognitive impairment, chemotherapy-related cognitive impairment, epilepsy, Angelman syndrome, stroke, diabetes induced neuropathy, Down's syndrome, autism, essential tremor, fronto-temporal dementia, progressive supranuclear palsy, amyotrophic lateral sclerosis, Huntington's disease, age associated memory impairment, chronic traumatic encephalopathy, cerebrovascular disease, Lewy' Body disease, or multiple system atrophy. In some embodiments, when the disease or condition is a neurological disorder, the compound or salt thereof is an agonist of nuclear receptor TLX (NR2E1).

In some embodiments, the cancer is glioblastoma, neuroblastoma, retinoblastoma, breast cancer, prostate cancer, or pancreatic cancer. In some embodiments, when the disease or condition is cancer, the compound or salt thereof is an antagonist of nuclear receptor TLX (NR2E1) or an inverse agonist nuclear receptor TLX (NR2E1).

In some embodiments, the retinopathy and/or metabolic disorder is diabetic retinopathy or diabetes. In some embodiments, when the disease or condition is a retinopathy and/or metabolic disorder, the compound or salt thereof is an agonist of nuclear receptor TLX (NR2E1).

In another aspect, the present methods are for modulating activity of nuclear receptor TLX (NR2E1). The methods comprise contacting nuclear receptor TLX (NR2E1) with a compound according to Formulas (I), (II) or (Ill), or a pharmaceutically acceptable salt thereof. As used herein, the terms “contacting” or “contact” refer to enabling or allowing a compound to become sufficiently close to react, interact, or physically touch nuclear receptor TLX (NR2E1). Such contact can occur in vivo, in vitro or ex vivo.

In another aspect, the present methods are for promoting neurogenesis and/or neural stem cell proliferation. The methods can comprise contacting a neural stem cell (NSC), a neuronal progenitor cell, a neuroblast, an immature neuron, a mature neuron, or a mixture thereof with a compound or salt thereof as described herein, in an amount effective to promote neurogenesis or NSC proliferation. Neurogenesis generally refers to the process by which neurons are created. Neurogenesis encompasses proliferation of neural stem cells, differentiation of cells into new neural cell types such as neuronal progenitor cell and neuroblasts, and/or migration and survival of the new cells. Neurogenesis is promoted when one or more stages of the process are initiated, increased, or accelerated, including but not limited to increasing proliferation or initiating differentiation. Neurogenesis may be desired in response to disease, damage or therapeutic intervention. An amount effective of the compound to promote neurogenesis is an amount of a compound described herein effective to achieve a statistically significant promotion of neurogenesis compared to the absence of the compound, or to achieve a desired fold-increase compared to the absence of the compound. In some embodiments, the methods promote neurogenesis by increasing neural stem cell proliferation by at least 2-fold, at least 10-fold, or at least 50-fold, or more, compared to the absence of the compound.

Neurogenesis or NSC proliferation may be at the level of a cell or tissue. The cell or tissue may be present in a subject or in an in vitro or ex vivo setting. In some embodiments, neurogenesis is stimulated or increased in a cell or tissue, such as that of the central or peripheral nervous system of a subject.

In certain embodiments, the compounds or salts thereof for use in the present methods are provided in a pharmaceutical composition. Such pharmaceutical compositions comprises one or more fatty acid mimetics, optionally together with one or more inert carriers and/or diluents.

### E. Methods of Conformation Profiling of Ligand Binding to Nuclear Receptor TLX (NR2E1)

In another aspect, this disclosure provides methods of conformational profiling of ligand binding to nuclear receptor TLX (NR2E1) as a means to narrow the conformational space relevant for identifying or designing ligands for nuclear receptor TLX (NR2E1). The method can comprise contacting one or more fatty acid mimetics with nuclear receptor TLX (NR2E1); and assessing function of each of the fatty acid mimetics as determined by the resulting activity of nuclear receptor TLX (NR2E1). In some embodiments, the method also comprises making a set of fatty acid mimetics prior to the contacting step.

The information gleaned from this profiling can identify subpopulations within the set of fatty acid mimetics that have structural features that are preferred for achieving the desired function with respect to nuclear receptor TLX (NR2E1). Further structural rigidification of the identified fatty acid mimetics is appreciated as a means to optimize ligands based on such conformational profiling.

The fatty acid mimetics of the set will have a plurality of different structures. In some embodiments, the set has at least 6 different structures, or at least 12, 24, 36, 48 or 96 different structures. In some embodiments, each of the fatty acid mimetics is a mimetic of (a) a C12 fatty acid; (b) a C14 fatty acid; (c) a C16 fatty acid; (d) a C18 fatty acid; (e) a C20 fatty acid; or (f) a C22 fatty acid. For example, where each of the mimetics of the set is a C18 fatty acid mimetic, the set can comprise each of Compounds 1-A to 4-F.

In some embodiments of the kits or sets, each of the fatty and acid mimetics is in a separate vessel. The vessel(s) in which the fatty and acid mimetics are supplied can be any conventional container that is capable of holding the supplied form, for instance, microfuge tubes, microtiter plates, ampules, bottles, or integral testing devices, such as fluidic devices, cartridges, lateral flow, or other similar devices.

The kits or sets can also include packaging materials for holding the vessels or combination of vessels. Typical packaging materials for such kits and systems include solid matrices (e.g., glass, plastic, paper, foil, micro-particles and the like) that hold the fatty and acid mimetics in any of a variety of configurations (e.g., in a vial, microtiter plate well, microarray, and the like). The kits may further include instructions recorded in a tangible form for use of the fatty and acid mimetics.

In some embodiments, each of the fatty acid mimetics is conformationally constrained due to their precise molecular structures, and each of the mimetics in the set has a different structure from each of the other mimetics in the set. The kits or sets can have any number of fatty acid mimetics, such as at least 4, 6, 12 or 24 or more fatty acid mimetics. In some embodiments, each of the fatty acid mimetics is a mimetic of (a) a C12 fatty acid; (b) a C14 fatty acid; (c) a C16 fatty acid; (d) a C18 fatty acid; (e) a C20 fatty acid; or (f) a C22 fatty acid. As an example, a kit or set of C18 fatty acid mimetics can include each of Compounds 1-A to 4-F in separate vessels.

The function of a compound or salt described herein (such as the fatty acid mimetics) as a modulator of nuclear receptor TLX (NR2E1) can be assessed by any suitable technique. In some embodiments, a compound is assessed in an assay suitable for determining the compound's function as an agonist or an antagonist of nuclear receptor TLX (NR2E1). Exemplary assays are described in Griffett et al., Cell Chem Biol 2020 Oct. 15; 27(10):1272-1284.e4.

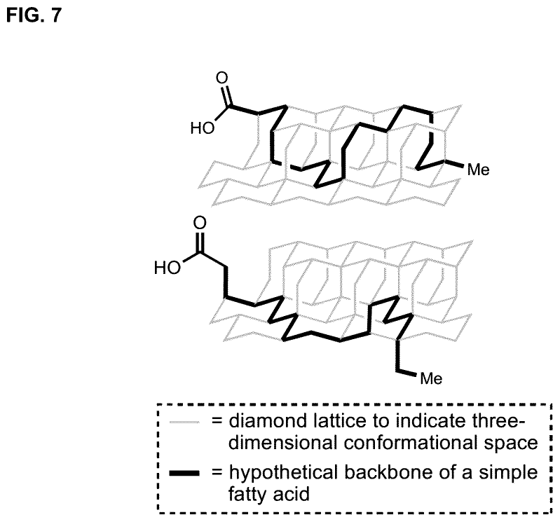

The present method can also comprise creating a model that shows conformation of the target fatty acid bound to nuclear receptor TLX (NR2E1). The model is based on the assessment of which of the mimetics of the set induced the desired activity of nuclear receptor TLX (NR2E1). For example, the model can comprise a lattice that depicts the three-dimensional space of the target fatty acid bound to nuclear receptor TLX (NR2E1). An exemplary lattice that depicts two potential three-dimensional conformations of oleic acid is shown in FIG. 7.

### F. Exemplary Embodiments

Exemplary Embodiment 1. A method for treating a disease or condition associated with nuclear receptor TLX (NR2E1) in a subject in need thereof, the method comprising administering to the subject a therapeutically effective amount of a compound of Formula (I), Formula (II), Formula (III), or a pharmaceutically acceptable salt thereof:

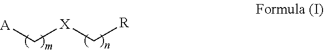

wherein:
A comprises a polar head group;
X in Formula (I) comprises a conformationally constrained core comprising two or more conformational constraints and at least one optional flexible subunit;
R comprises a non-polar group; and
each of m and n is independently an integer from 1 to 25, or from 1 to 19, or from 1 to 11, or from 1 to 7;

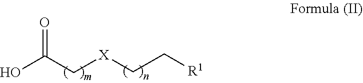

wherein:
X in Formula (II) comprises a constrained core comprising one or more acyclic conformational constraints and at least one optional flexible subunit;
R¹ is a C₁₋₆-alkyl; and
each of m and n is independently an integer from 1 to 25, or from 1 to 19, or from 1 to 11, or from 1 to 7;
provided that the compound is not a natural product and/or
provided that at least one conformational constraint is a conformational constraint other than

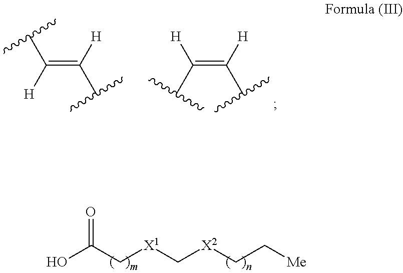

wherein:
X¹ is

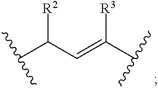

R² is C₁₋₆-alkyl;
R³ is C₁₋₆-alkyl;
X² is

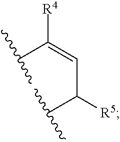

R⁴ is C₁₋₆-alkyl;
R⁵ is hydrogen, deuterium, or C₁₋₆-alkyl; and
each of m and n is independently an integer from 1 to 25, or from 1 to 19, or from 1 to 11, or from 1 to 7.

Exemplary Embodiment 2. The method of embodiment 1, wherein the disease or condition associated with nuclear receptor TLX (NR2E1) is a neurological disease or condition.

Exemplary Embodiment 3. The method of embodiment 1, wherein the disease or condition associated with nuclear receptor TLX (NR2E1) comprises dementia and/or cognitive dysfunction.

Exemplary Embodiment 4. The method of embodiment 1, wherein the disease or condition associated with nuclear receptor TLX (NR2E1) is Alzheimer's disease. Exemplary Embodiment 5. A method for modulating activity of nuclear receptor TLX (NR2E1), the method comprising contacting nuclear receptor TLX (NR2E1) with a compound of Formula (I), Formula (II), Formula (III), or a pharmaceutically acceptable salt thereof.

Exemplary Embodiment 6. A method for promoting neurogenesis and/or neural stem cell proliferation, the method comprising contacting a cell with a compound of Formula (I), Formula (II), Formula (III), or a pharmaceutically acceptable salt thereof.

Exemplary Embodiment 7. The method of embodiment 5 or claim 6, wherein said contacting occurs in vitro.

Exemplary Embodiment 8. The method of embodiment 5 or claim 6, wherein said contacting occurs in vivo.

Exemplary Embodiment 9. The method of embodiment 8, wherein said contacting occurs in vivo in a subject having, or at risk of having, dementia and/or cognitive dysfunction.

Exemplary Embodiment 10. The method of embodiment 8, wherein said contacting occurs in vivo in a subject having, or at risk of having, Alzheimer's disease.

Exemplary Embodiment 11. The compound of Formula (I), Formula (II), Formula (III), or a pharmaceutically acceptable salt thereof for use in the treatment of a disease or condition associated with nuclear receptor TLX (NR2E1).

Exemplary Embodiment 12. The compound or pharmaceutically acceptable salt of embodiment 11, wherein the disease or condition associated with nuclear receptor TLX (NR2E1) is a neurological disease or condition.

Exemplary Embodiment 13. The compound or pharmaceutically acceptable salt of embodiment 11, wherein the disease or condition associated with nuclear receptor TLX (NR2E1) involves dementia and/or cognitive dysfunction.

Exemplary Embodiment 14. The compound or pharmaceutically acceptable salt of embodiment 11, wherein the disease or condition associated with nuclear receptor TLX (NR2E1) is Alzheimer's disease.

Exemplary Embodiment 15. The method of embodiments 1-10 or the compound or pharmaceutically acceptable salt of embodiments 11-14, wherein the compound is selected from one of the following compounds:

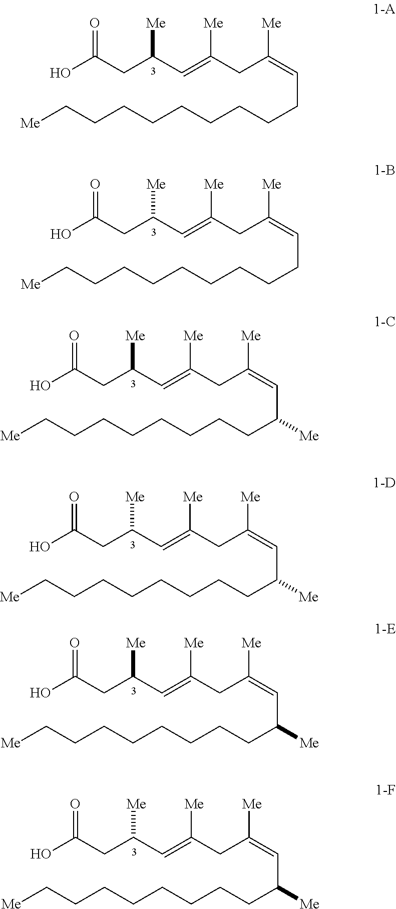

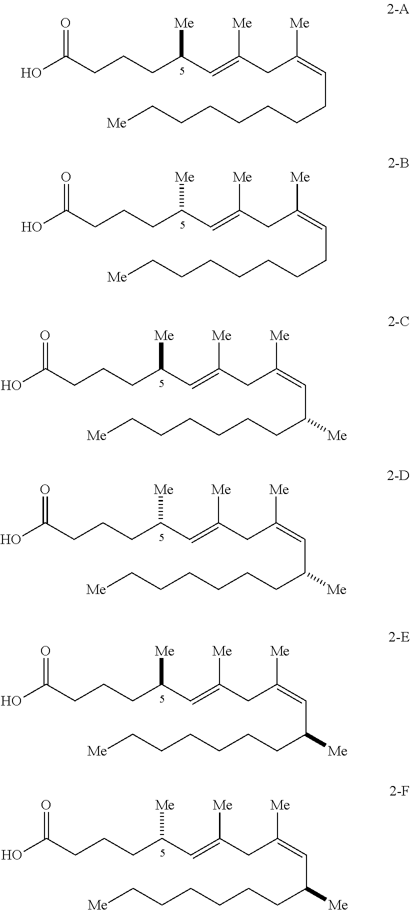 [SMILES: CCCCCCCC/C=C(/C)C/C(C)=C/[C@H](C)CCCC(=O)O ; CCCCCCCC/C=C(/C)C/C(C)=C/[C@@H](C)CCCC(=O)O ; CCCCCCC[C@@H](C)/C=C(/C)C/C(C)=C/[C@H](C)CCCC(=O)O ; CCCCCCC[C@@H](C)/C=C(/C)C/C(C)=C/[C@@H](C)CCCC(=O)O ; CCCCCCC[C@H](C)/C=C(/C)C/C(C)=C/[C@H](C)CCCC(=O)O ; CCCCCCC[C@H](C)/C=C(/C)C/C(C)=C/[C@@H](C)CCCC(=O)O]

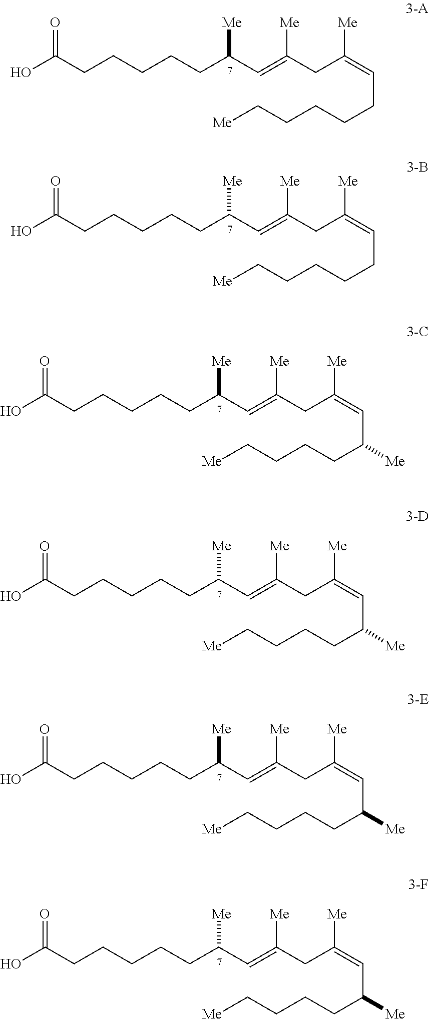 [SMILES: CCCCCC/C=C(/C)C/C(C)=C/[C@H](C)CCCCCC(=O)O ; CCCCCC/C=C(/C)C/C(C)=C/[C@@H](C)CCCCCC(=O)O ; CCCCC[C@@H](C)/C=C(/C)C/C(C)=C/[C@H](C)CCCCCC(=O)O ; CCCCC[C@@H](C)/C=C(/C)C/C(C)=C/[C@@H](C)CCCCCC(=O)O ; CCCCC[C@H](C)/C=C(/C)C/C(C)=C/[C@H](C)CCCCCC(=O)O ; CCCCC[C@H](C)/C=C(/C)C/C(C)=C/[C@@H](C)CCCCCC(=O)O]

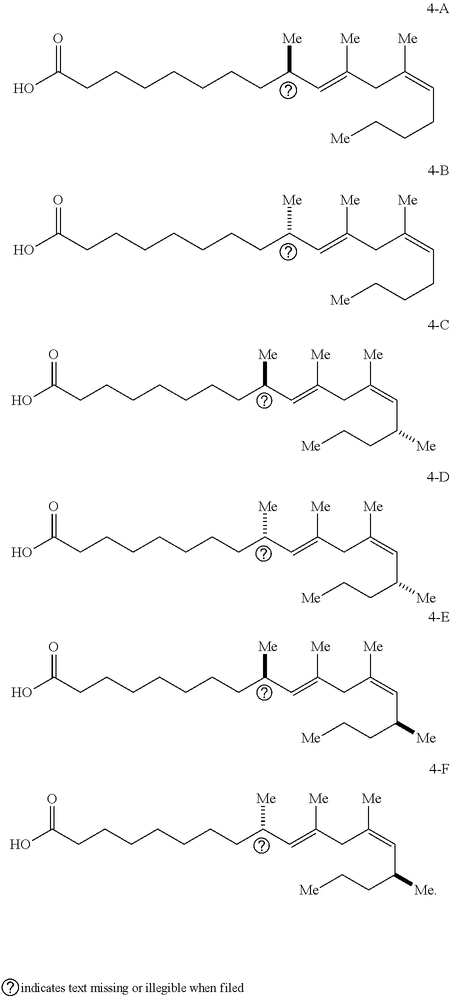 [SMILES: CCCC/C=C(/C)C/C(C)=C/[C@H](C)CCCCCCCC(=O)O ; CCCC/C=C(/C)C/C(C)=C/[C@@H](C)CCCCCCCC(=O)O ; CCC[C@@H](C)/C=C(/C)C/C(C)=C/[C@H](C)CCCCCCCC(=O)O ; CCC[C@@H](C)/C=C(/C)C/C(C)=C/[C@@H](C)CCCCCCCC(=O)O ; CCC[C@H](C)/C=C(/C)C/C(C)=C/[C@H](C)CCCCCCCC(=O)O ; CCC[C@H](C)/C=C(/C)C/C(C)=C/[C@@H](C)CCCCCCCC(=O)O]

Exemplary Embodiment 16. The method of embodiments 1-10 or the compound or pharmaceutically acceptable salt of embodiments 11-14, wherein the flexible subunit of Formula (I) is a methylene subunit.

Exemplary Embodiment 17. The method of embodiments 1-10 or the compound or pharmaceutically acceptable salt of embodiments 11-14, wherein X in Formula (I) comprises (ix)

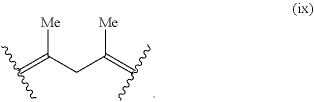 [SMILES: *=C(C)CC(=*)C]

Exemplary Embodiment 18. The method of embodiments 1-10 or the compound or pharmaceutically acceptable salt of embodiments 11-14, wherein each of the two or more conformational constraints in Formula (I) is independently selected from the group consisting of (x), (x′), (x″), and (x″′):

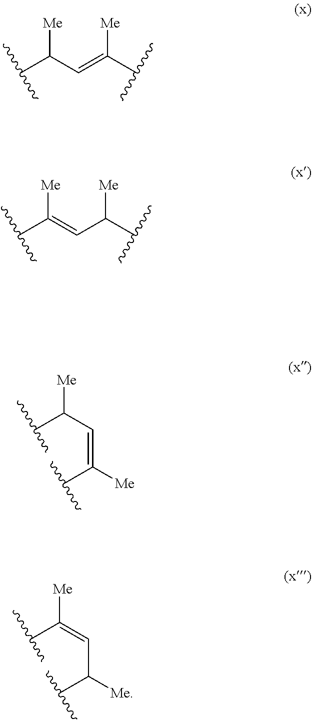 [SMILES: */C(C)=C/C(*)C ; */C(C)=C/C(*)C ; */C(C)=C\C(*)C ; */C(C)=C\C(*)C]

Exemplary Embodiment 19. The method or compound or pharmaceutically acceptable salt of embodiment 18, wherein the two or more conformational constraints are joined by a C₁₋₆-alkylene, such as a C₁₋₃-alkylene (e.g., methylene, ethylene, or propylene), preferably C₁ (e.g., methylene).

Exemplary Embodiment 20. The method of embodiments 1-10 or the compound or pharmaceutically acceptable salt of embodiments 11-14, wherein A comprises A²-R¹⁵—, wherein A² is a polar head group, and R¹⁵ is a bond, a C₁₋₆-alkyl, or a C₂-C₆ alkenyl.

Exemplary Embodiment 21. The method or compound or salt of embodiment 20, wherein A² is a carboxylic acid, a carboxyl ester, an amide, a ketone, a phosphonic acid, a phosphinic acid, a sulfonic acid, a sulfinic acid, a sulfonamide, an acyl-sulfonamide, a hydroxamic acid, a hydroxamic ester, a sulfonylurea, an acylurea, a tetrazole, a thiazolidinedione, an oxazolidinedione, an oxadiazolone, a thiadiazolone, an oxathiadiazole oxide, an oxadiazolethione, an isoxazole, a tetramic acid, a cyclopentane diones, a phenol derivative, a squaric acid derivative, or a salt thereof.

Exemplary Embodiment 22. The method or compound or salt of embodiment 21, wherein the constrained core in the compound of Formula (II) comprises two or more acyclic conformational constraints.

Exemplary Embodiment 23. The method or compound or salt of embodiment 21 or claim 22, wherein each of the one or more acyclic conformational constraints in Formula (II) is (x), (x′), (x″), or (x″′):

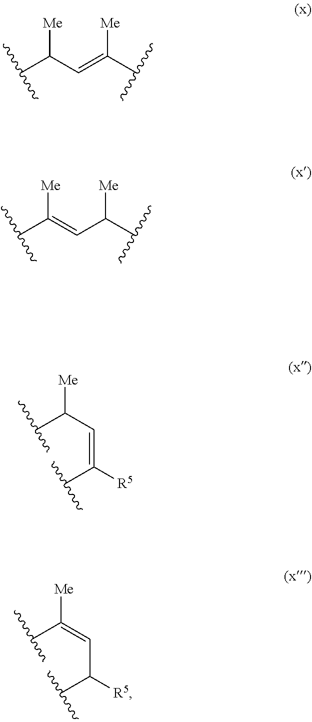

wherein R⁵ is hydrogen or C₁₋₆-alkyl.

Exemplary Embodiment 24. The method or compound or salt of any one of embodiments 21-23, wherein each acyclic conformational constraint in Formula (II) is joined by a C₁₋₆-alkylene, such as a C₁₋₃-alkylene (e.g., methylene, ethylene, or propylene), preferably C₁ (e.g., methylene).

Exemplary Embodiment 25. The method or compound or salt of any one of embodiments 21-23, wherein X in Formula (II) comprises:

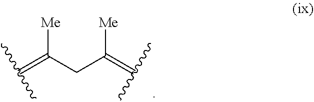 [SMILES: *=C(C)CC(=*)C]

Exemplary Embodiment 26. The method or compound or salt of embodiment 21, wherein X in Formula (II) is:

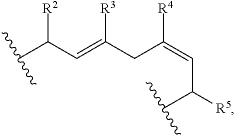

wherein:
R² is C₁₋₆-alkyl;
R³ is C₁₋₆-alkyl;
R⁴ is C₁₋₆-alkyl; and
R⁵ is hydrogen or C₁₋₆-alkyl.

Exemplary Embodiment 27. The method of embodiments 1-10 or the compound or salt of embodiments 11-14, wherein
X¹ in Formula (III) is (x.1) or (x.2):

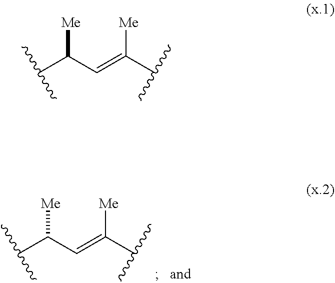 [SMILES: */C(C)=C/[C@@H](*)C ; */C(C)=C/[C@H](*)C]

X² in Formula (III) is (x″′.1) or (x″′.2):

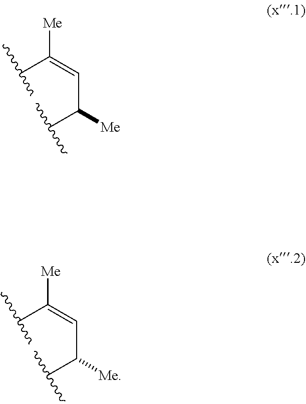 [SMILES: */C(C)=C\[C@H](*)C ; */C(C)=C\[C@@H](*)C]

Exemplary Embodiment 28. The method of embodiments 1-10 or the compound or salt of embodiments 11-14, wherein each of R², R³, and R⁴ are methyl or a partially or fully deuterated methyl, and R⁵ is hydrogen, deuterium, methyl, or a partially or fully deuterated methyl.

Exemplary Embodiment 29. The method of embodiments 1-10 or the compound or salt of embodiments 11-14, wherein each of R², R³, and R⁴ are methyl or a partially or fully deuterated methyl, and R⁵ is hydrogen or deuterium.

Exemplary Embodiment 30. The method of embodiments 1-10 or the compound or salt of embodiments 11-14, wherein each of R², R³, and R⁴ are methyl or a partially or fully deuterated methyl, and R⁵ is C₁₋₆-alkyl.

Exemplary Embodiment 31. The method of embodiments 1-10 or the compound or salt of embodiments 11-14, wherein each of R², R³, R⁴, and R⁵ are methyl or a partially or fully deuterated methyl.

Exemplary Embodiment 32. The method or compound or salt of any one of the preceding claims, wherein m is 1, 3, 5, 7, 9, 11, 13, 15, 17, 19, 21, 23 or 25.

Exemplary Embodiment 33. The method or compound or salt of any one of the preceding claims, wherein n is 1, 3, 5, 7, 9, 11, 13, 15, 17, 19, 21, 23 or 25.

Exemplary Embodiment 34. The method or compound or salt of any one of the preceding claims, wherein m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40.

Exemplary Embodiment 35. The method of embodiments 1-10 or the compound or salt of embodiments 11-14, wherein the compound has a structure of Formula (I-A):

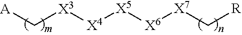

wherein X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.

Exemplary Embodiment 36. The method or compound or salt of embodiment 35, wherein at least one of X³ or X⁷ is a conformational constraint.

Exemplary Embodiment 37. The method or compound or salt of embodiment 35, wherein at least one of X³ or X⁷ is selected from (i) to (x):

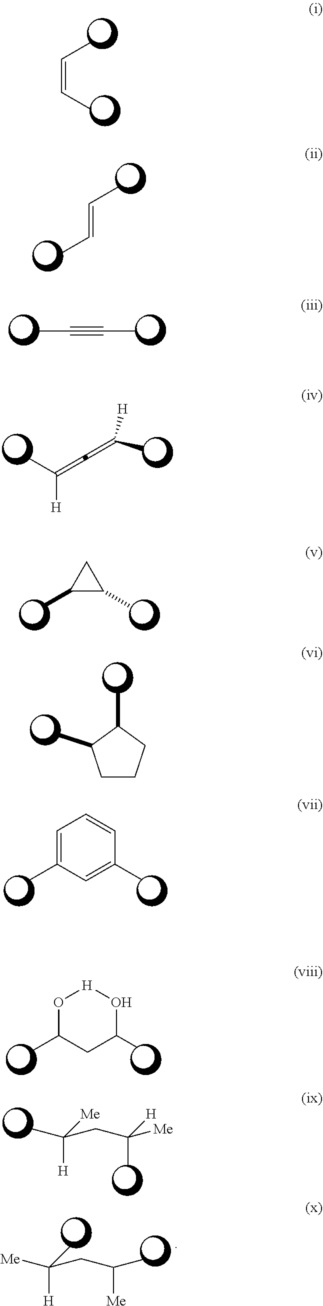

Exemplary Embodiment 38. The method or compound or salt of embodiment 35, wherein at least one of X³ or X⁷ is a conformational constraint comprising a ring system.

Exemplary Embodiment 39. The method or compound or salt of embodiment 38, wherein at least one of X³ or X⁷ is a cycloalkyl.

Exemplary Embodiment 40. The method or compound or salt of embodiment 39, wherein at least one of X³ or X⁷ is selected from a stereodefined disubstituted cyclopentane, cyclopropane, cyclohexane, tetrahydrofuran, tetrahydropyran, g-lactone, g-lactam, regiodefined di- or tri-substituted pyrone, pyridone, pyridine, or benzene.

Exemplary Embodiment 41. The method or compound or salt of any one of embodiments 35-40, wherein X⁴ and X⁶ are independently C₁₋₆-alkylene, such as a C₁₋₃-alkylene (e.g., methylene, ethylene, or propylene), preferably C₁ (e.g., methylene).

Exemplary Embodiment 42. A method of conformational profiling of ligand binding to nuclear receptor TLX (NR2E1) comprising:
preparing a set of mimetics of the target fatty acid; and assessing the functional activity of each member of the set of fatty acid mimetics at nuclear receptor TLX (NR2E1).

Exemplary Embodiment 43. The method of embodiment 42, further comprising identifying one or more subpopulations of the set that have structural features that are preferred for modulating activity of nuclear receptor TLX (NR2E1).

Exemplary Embodiment 44. The method of embodiment 43, further comprising preparing a subset of fatty acid mimetics comprising the structural features of the one or more subpopulations, wherein the mimetics of the subset comprise additional structural rigidification compared to the mimetics of the subpopulation.

Exemplary Embodiment 45. The method of embodiment 42, wherein the mimetics of the set have at least 6 different structures.

Exemplary Embodiment 46. The method of embodiment 42, wherein the mimetics of the set have at least 24 different structures.

Exemplary Embodiment 47. The method of any one of embodiments 42-46, wherein each of the fatty acid mimetics is a mimetic of:
(a) a C₁₂ fatty acid;
(b) a C₁₄ fatty acid;
(c) a C₁₆ fatty acid;
(d) a C₁₈ fatty acid;
(e) a C₂₀ fatty acid; or
(f) a C₂₂ fatty acid.

Exemplary Embodiment 48. The method of any one of embodiments 42-47, wherein each of the mimetics of the set is a C18 fatty acid mimetic, and the set comprises each of Compounds 1-A to 4-F:

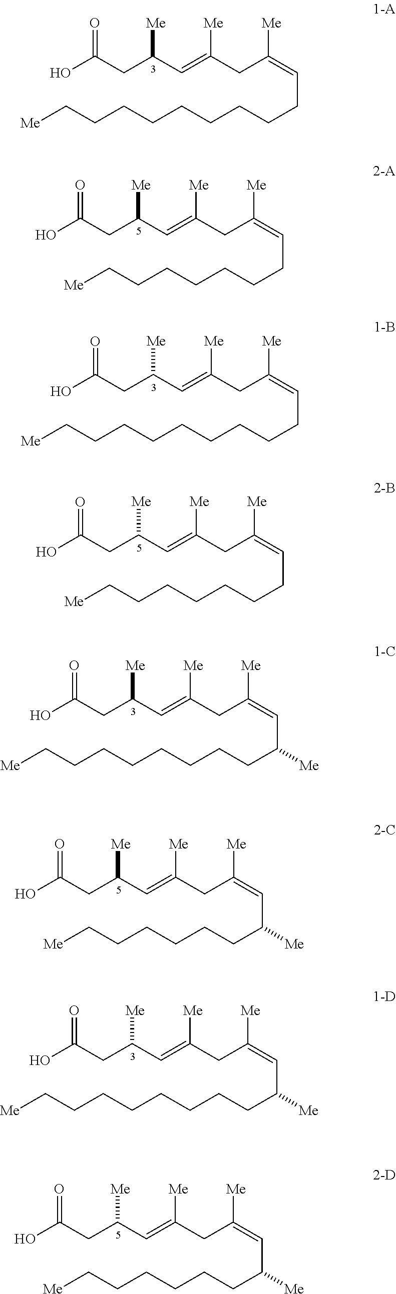

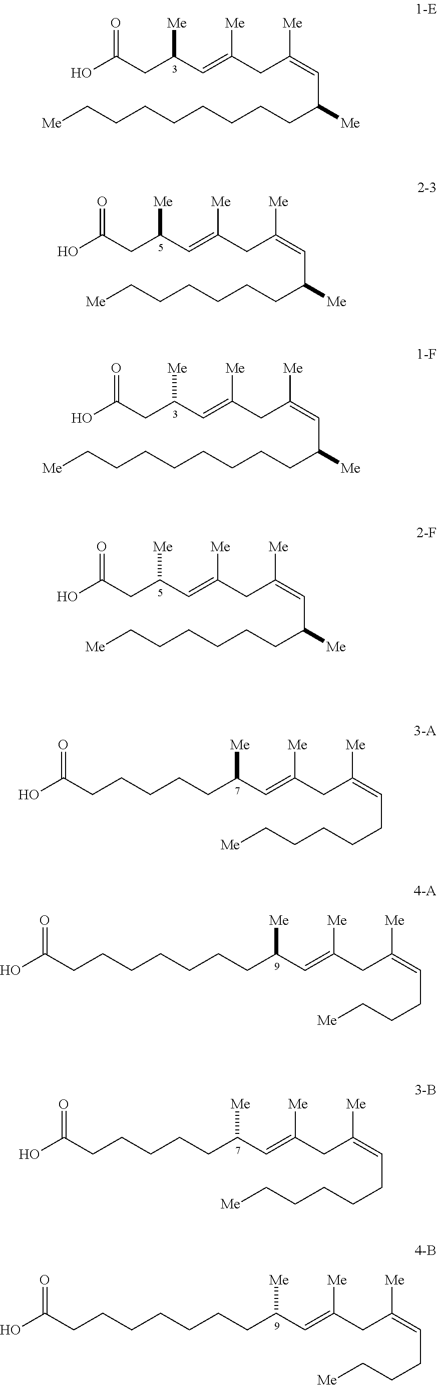

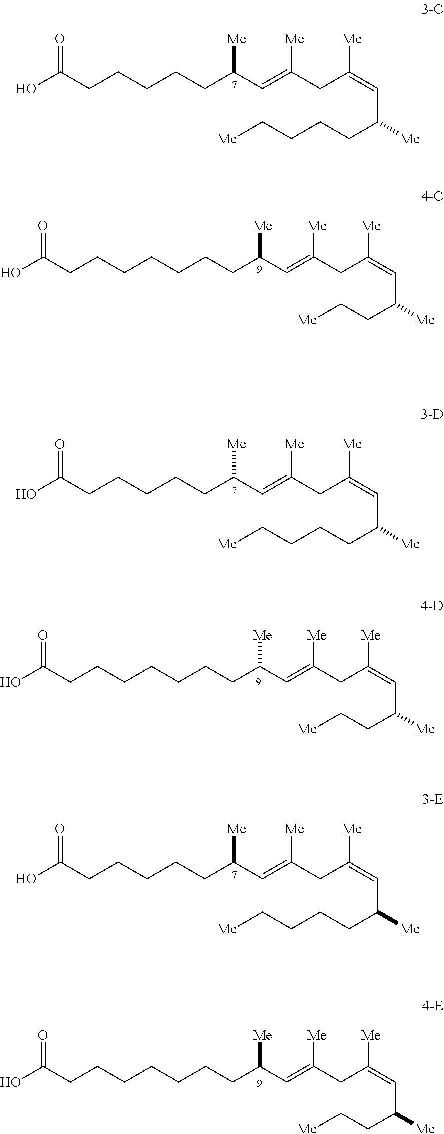 [SMILES: CCCCC[C@@H](C)/C=C(/C)C/C(C)=C/[C@H](C)CCCCCC(=O)O ; CCC[C@@H](C)/C=C(/C)C/C(C)=C/[C@H](C)CCCCCCCC(=O)O ; CCCCC[C@@H](C)/C=C(/C)C/C(C)=C/[C@@H](C)CCCCCC(=O)O ; CCC[C@@H](C)/C=C(/C)C/C(C)=C/[C@@H](C)CCCCCCCC(=O)O ; CCCCC[C@H](C)/C=C(/C)C/C(C)=C/[C@H](C)CCCCCC(=O)O ; CCC[C@H](C)/C=C(/C)C/C(C)=C/[C@H](C)CCCCCCCC(=O)O]

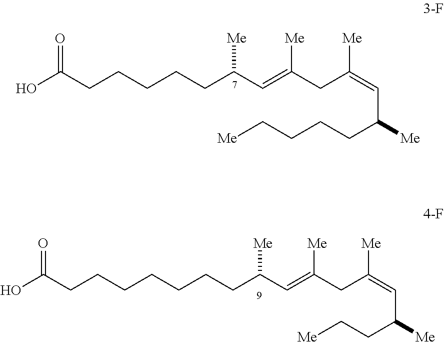 [SMILES: CCCCC[C@H](C)/C=C(/C)C/C(C)=C/[C@@H](C)CCCCCC(=O)O ; CCC[C@H](C)/C=C(/C)C/C(C)=C/[C@@H](C)CCCCCCCC(=O)O]

Exemplary Embodiment 49. The method of any one of embodiments 41-48, further comprising creating a model that shows conformation of the target fatty acid bound to nuclear receptor TLX (NR2E1), wherein the model is based on the assessment of which of the mimetics of the set bound to nuclear receptor TLX (NR2E1).

Exemplary Embodiment 50. The method of embodiment 49, wherein the model comprises a lattice that depicts the three-dimensional space of the target fatty acid bound to nuclear receptor TLX (NR2E1).

### G. EXAMPLES

### Example 1

In this example, four different classes of oleic acid mimetics (termed Class I-IV) were prepared using the synthetic schemes set forth herein. Each class differs by the position of the conformational biasing element in the C₁₈ backbone. The carbon number where each conformational constraint begins within each ligand class is specified. The set of compounds is depicted in Table 2 and includes six different compounds for each mimetic “Class” (1A-F, 2A-F, 3A-F, and 4A-F).

TABLE 2
Class I:

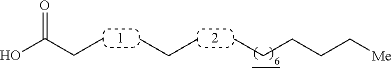

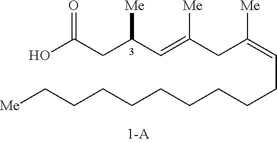 [SMILES: CCCCCCCCCC/C=C(/C)C/C(C)=C/[C@H](C)CC(=O)O]

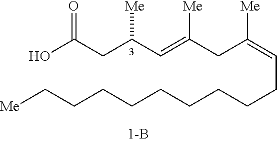 [SMILES: CCCCCCCCCC/C=C(/C)C/C(C)=C/[C@@H](C)CC(=O)O]

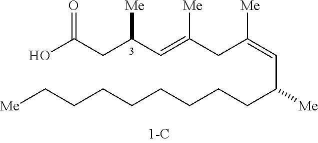 [SMILES: CCCCCCCCC[C@@H](C)/C=C(/C)C/C(C)=C/[C@H](C)CC(=O)O]

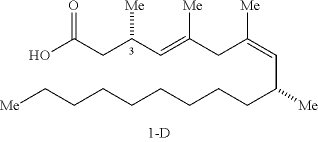 [SMILES: CCCCCCCCC[C@@H](C)/C=C(/C)C/C(C)=C/[C@@H](C)CC(=O)O]

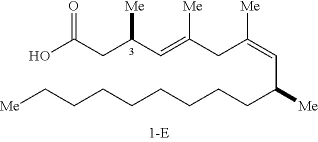 [SMILES: CCCCCCCCC[C@H](C)/C=C(/C)C/C(C)=C/[C@H](C)CC(=O)O]

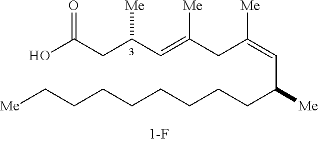 [SMILES: CCCCCCCCC[C@H](C)/C=C(/C)C/C(C)=C/[C@@H](C)CC(=O)O]

Class II:

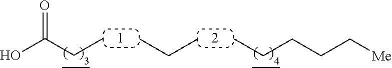

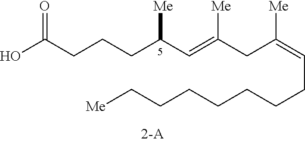 [SMILES: CCCCCCCC/C=C(/C)C/C(C)=C/[C@H](C)CCCC(=O)O]

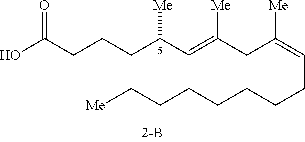 [SMILES: CCCCCCCC/C=C(/C)C/C(C)=C/[C@@H](C)CCCC(=O)O]

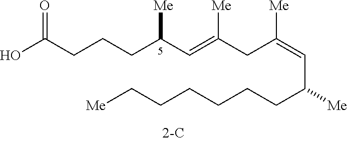 [SMILES: CCCCCCC[C@@H](C)/C=C(/C)C/C(C)=C/[C@H](C)CCCC(=O)O]

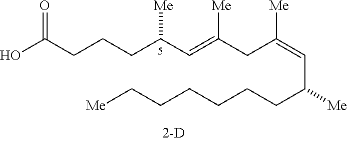 [SMILES: CCCCCCC[C@@H](C)/C=C(/C)C/C(C)=C/[C@@H](C)CCCC(=O)O]

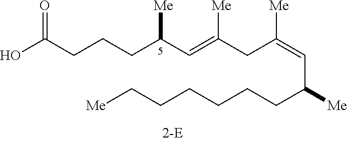 [SMILES: CCCCCCC[C@H](C)/C=C(/C)C/C(C)=C/[C@H](C)CCCC(=O)O]

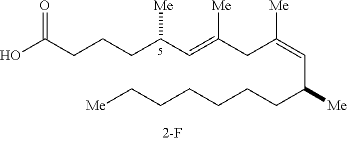 [SMILES: CCCCCCC[C@H](C)/C=C(/C)C/C(C)=C/[C@@H](C)CCCC(=O)O]

Class III:

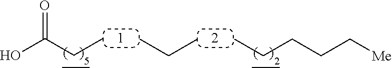

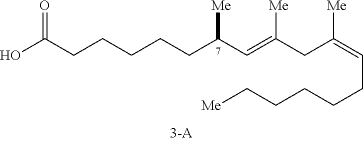 [SMILES: CCCCCC/C=C(/C)C/C(C)=C/[C@H](C)CCCCCC(=O)O]

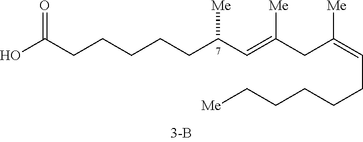 [SMILES: CCCCCC/C=C(/C)C/C(C)=C/[C@@H](C)CCCCCC(=O)O]

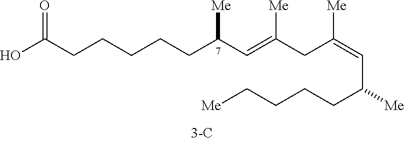 [SMILES: CCCCC[C@@H](C)/C=C(/C)C/C(C)=C/[C@H](C)CCCCCC(=O)O]

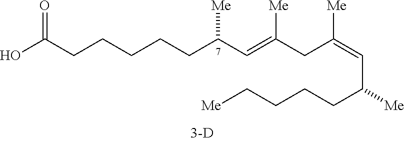 [SMILES: CCCCC[C@@H](C)/C=C(/C)C/C(C)=C/[C@@H](C)CCCCCC(=O)O]

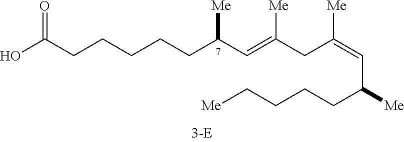 [SMILES: CCCCC[C@H](C)/C=C(/C)C/C(C)=C/[C@H](C)CCCCCC(=O)O]

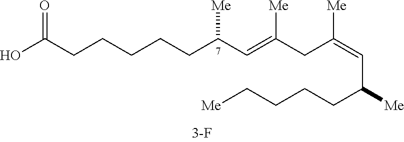 [SMILES: CCCCC[C@H](C)/C=C(/C)C/C(C)=C/[C@@H](C)CCCCCC(=O)O]

Class IV:

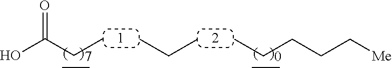

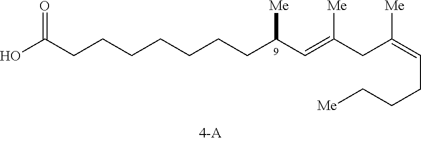 [SMILES: CCCC/C=C(/C)C/C(C)=C/[C@H](C)CCCCCCCC(=O)O]

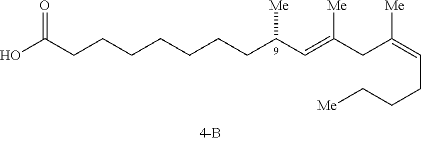 [SMILES: CCCC/C=C(/C)C/C(C)=C/[C@@H](C)CCCCCCCC(=O)O]

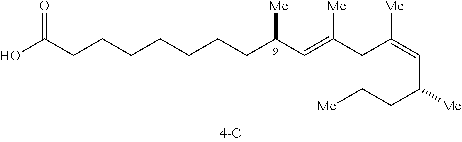 [SMILES: CCC[C@@H](C)/C=C(/C)C/C(C)=C/[C@H](C)CCCCCCCC(=O)O]

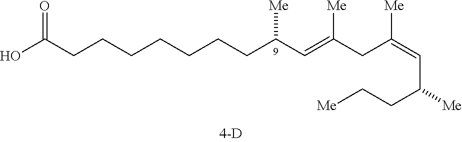 [SMILES: CCC[C@@H](C)/C=C(/C)C/C(C)=C/[C@@H](C)CCCCCCCC(=O)O]

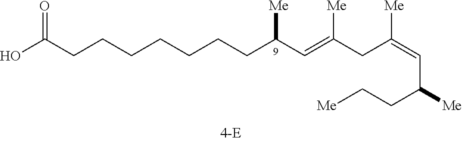 [SMILES: CCC[C@H](C)/C=C(/C)C/C(C)=C/[C@H](C)CCCCCCCC(=O)O]

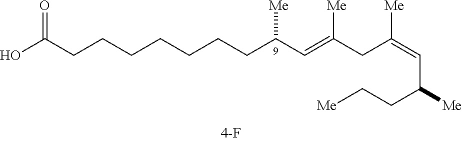 [SMILES: CCC[C@H](C)/C=C(/C)C/C(C)=C/[C@@H](C)CCCCCCCC(=O)O]

For Class 1 compounds, the chiral conformational constraint elements first appear at C3 of the fatty acid backbone, with the other classes moving the positioning of the constraints to begin at C5, C7 and C9, respectively. Comparing the compounds across each Glass, ligands with the same alphabetical descriptor (e.g., “A,” vs “B”, vs “C”, etc.) share the same stereodefined constraint (same substitution and stereochemistry), but differ with respect to where the constraint is placed within the 18-carbon backbone.

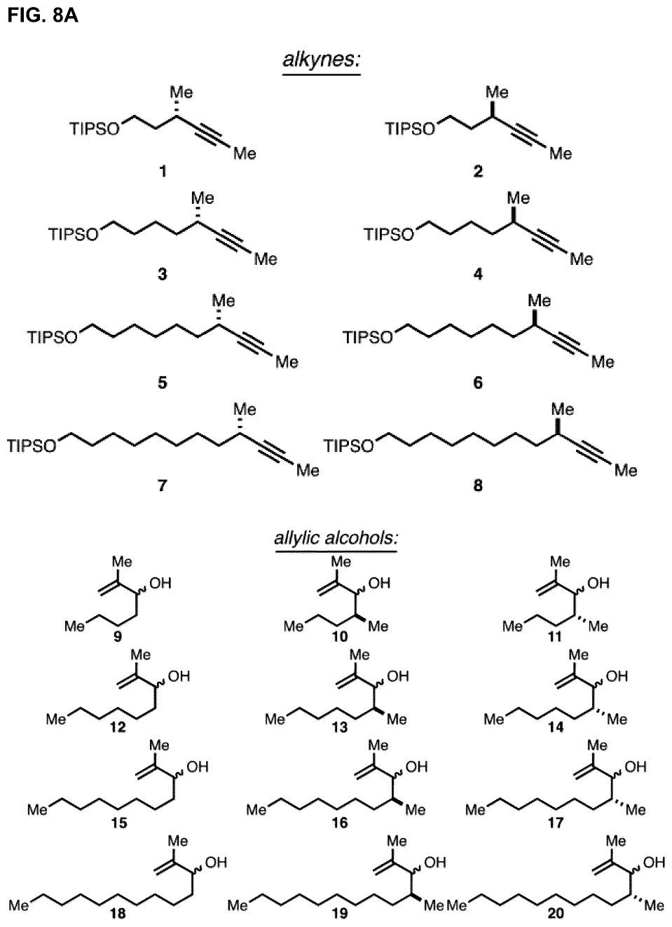

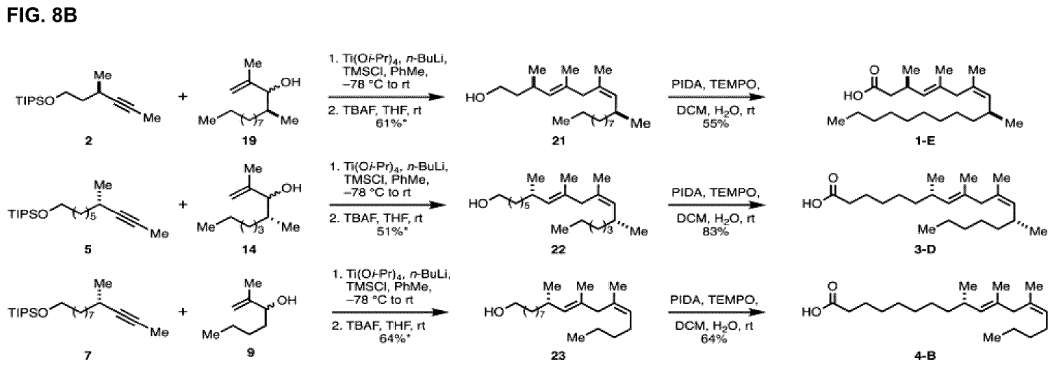

FIGS. 8A and 8B illustrate the convergent synthesis of members of the fatty acid mimetic collection. FIG. 8A shows structures of the alkynes and allylic alcohols employed.

FIG. 8B provides examples of the chemical pathway for synthesis of members of the fatty acid mimetic collection. The three-step process enabled stereoselective coupling and conversion to the designed fatty acid mimetics.

Yield includes all isomers after deprotection: 21-rs=3:1, Z:E (of major regioisomer)=13:1; 22-rs=4:1, Z:E (of major regioisomer)=10:1; 23-rs=4:1, Z:E (of major regioisomer)=7:1, only one alkene stereoisomer visible via ¹HNMR for the minor regioisomeric coupling product.

### Example 2

In these experiments, compounds from Example 1 were evaluated in a biochemical assay to characterize ligand binding to TLX by detecting alterations in the ability of the TLX ligand binding domain to recruit an NR box peptide derived from the nuclear receptor-interacting protein 1 (NRIP1; receptor-interacting protein 140 (RIP140) transcriptional cofactor. RIP140 has been previously identified as a TLX interacting protein (Corso-Diaz X, et al. (2016) Co-activator candidate interactions for orphan nuclear receptor NR2E1. BMC Genomics 17:832).

AlphaScreen (Perkin Elmer, Shelton, CT) technology was used to detect the TLX-RIP140 NR box peptide interaction. The AlphaScreen assay was performed as previously described using other nuclear receptors including ROR, FXR, LXR, VDR and RXR (Bettoun D J, et al., (2003) Mol Endocrinol 17:2320-2328; Bramlett K S, et al., (2003) Journal of Pharmacology and Experimental Therapeutics 307:291-296; Houck K A, at al., (2004) Mol Genet Metab 83:184-187; Kumar N, et al., (2010) Mol Pharmacol 77:228-236; and Wang Y, et al., (2010) ACS Chemical Biology: 1029-1034. The present experiments used the N-terminal his-tagged ligand-binding domain (LBD) of TLX (NovAliX SAS, Strasbourg, France) and an N-terminal biotinylated RIP140-L6 peptide with a N-terminal KGG linker, namely (KGGQDTSKNSKLNSHQKVTLLQLLLGHKNEENV (aa484-513)).

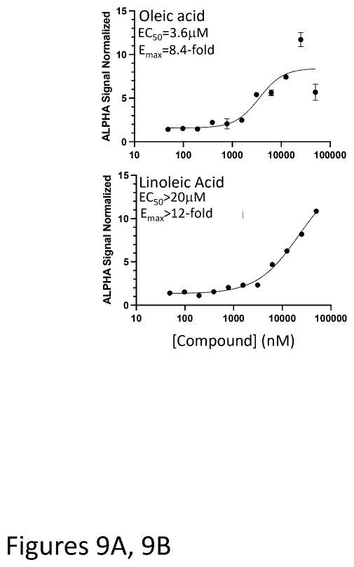

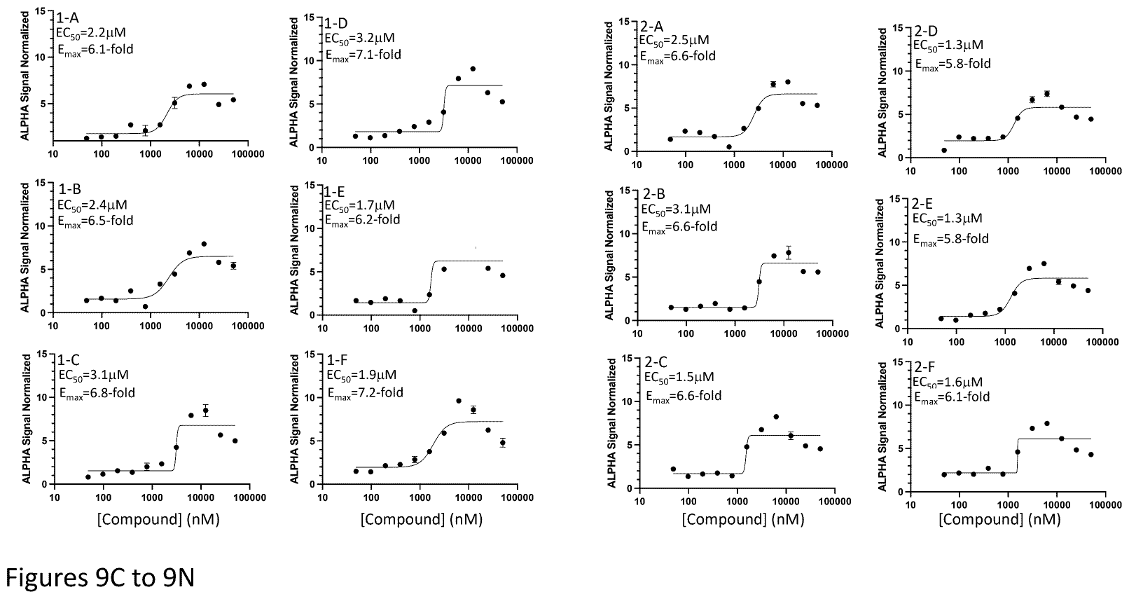

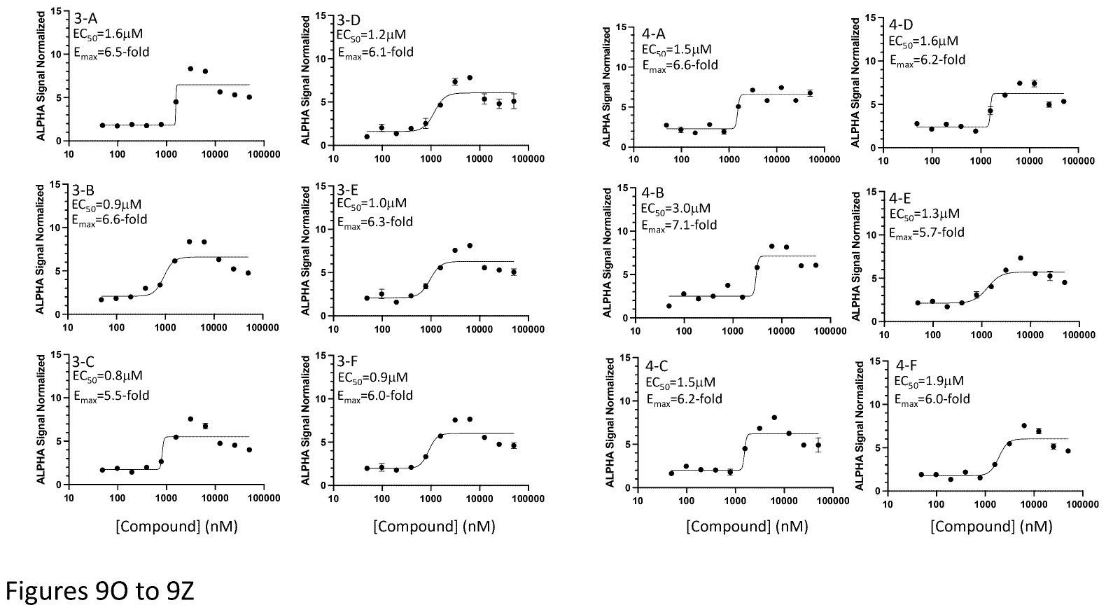

FIGS. 9A to 9Z show the measurements for oleic acid, linoleic acid, and Compounds 1-A to 4-F in the AlphaScreen assay.

Table 3 provides data from the experiments, including a comparison of the EC₅₀ values for Compounds 1-A to 4-F at nuclear receptor TLX (NR2E1).

TABLE 3

| | EC50 | Efficacy (fold | Relative |
|---|---|---|---|
| Compound | (μM) | increase) | efficacy (%) |
| Oleic Acid | 3.6 | 8.4 | 100.0 |
| 1-A | 2.2 | 6.1 | 72.6 |
| 1-B | 2.4 | 6.5 | 77.4 |
| 1-C | 3.1 | 6.8 | 81.0 |
| 1-D | 3.2 | 7.1 | 84.5 |
| 1-E | 1.7 | 6.2 | 73.8 |
| 1-F | 1.9 | 7.2 | 85.7 |
| 2-A | 2.5 | 6.6 | 78.6 |
| 2-B | 3.1 | 6.6 | 78.6 |
| 2-C | 1.5 | 6.6 | 78.6 |
| 2-D | 1.3 | 5.8 | 69.0 |
| 2-E | 1.3 | 5.8 | 69.0 |
| 2-F | 1.6 | 6.1 | 72.6 |
| 3-A | 1.6 | 6.5 | 77.4 |
| 3-B | 0.9 | 6.6 | 78.6 |
| 3-C | 0.8 | 5.5 | 65.5 |
| 3-D | 1.2 | 6.1 | 72.6 |
| 3-E | 1.0 | 6.3 | 75.0 |
| 3-F | 0.9 | 6.0 | 71.4 |
| 4-A | 1.5 | 6.6 | 78.6 |
| 4-B | 3.0 | 7.1 | 84.5 |
| 4-C | 1.5 | 6.2 | 73.8 |
| 4-D | 1.6 | 6.2 | 73.8 |
| 4-E | 1.3 | 5.7 | 67.9 |
| 4-F | 1.9 | 6.0 | 71.4 |

These compounds activate nuclear receptor TLX (NR2E1) with similar efficacy to oleic acid (65.5% to 85.7%).

The various compounds, materials, structures, methods, and systems are included by way of illustration and example only and not in any limiting sense. In view of this disclosure, the present teachings may be implemented in other applications and approaches, while remaining within the scope of the appended claims. Variations and modifications may be made to the embodiments described herein without substantially departing from the spirit and principles of the teachings described herein.

1. A method for modulating activity of nuclear receptor TLX (NR2E1), the method comprising contacting nuclear receptor TLX (NR2E1) with a compound of Formula (I), Formula (II), Formula (III), or a pharmaceutically acceptable salt thereof: 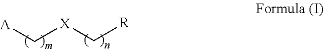
wherein:
A comprises a polar head group;
X in Formula (I) comprises a conformationally constrained core comprising two or more conformational constraints and at least one optional flexible subunit;
R comprises a non-polar group; and
each of m and n is independently an integer from 1 to 25, or from 1 to 19, or from 1 to 11, or from 1 to 7; 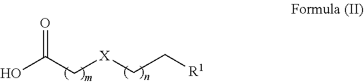
wherein:
X in Formula (II) comprises a constrained core comprising one or more acyclic conformational constraints and at least one optional flexible subunit;
R¹ is a C₁₋₆-alkyl; and
each of m and n is independently an integer from 1 to 25, or from 1 to 19, or from 1 to 11, or from 1 to 7;
provided that the compound is not a natural product and/or
provided that at least one conformational constraint is a conformational constraint other than 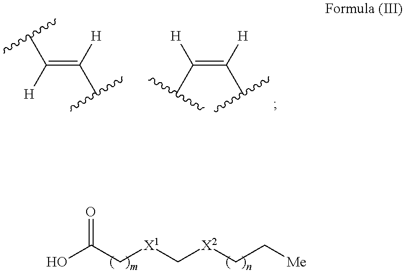
wherein:
X¹ is 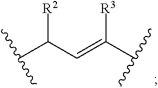
R² is C₁₋₆-alkyl;
R³ is C₁₋₆-alkyl;
X² is 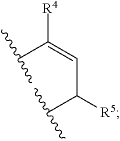
R⁴ is C₁₋₆-alkyl;
R⁵ is hydrogen, deuterium, or C₁₋₆-alkyl; and
each of m and n is independently an integer from 1 to 25, or from 1 to 19, or from 1 to 11, or from 1 to 7.

2. The method of claim 1, wherein said contacting occurs in vivo.

3. The method of claim 2, wherein said contacting occurs in vivo in a subject having, or at risk of having, dementia and/or cognitive dysfunction.

4. The method of claim 1, wherein m+n is 6, 7, 8, 9, 10, 11, 12, 13, 14, 15, 16, 17, 18, 19, 20, 21, 22, 23, 24 25, 26, 27, 28, 29, 30, 31, 32, 33, 34, 35, 36, 37, 38, 39, or 40.

5. The method of claim 1, wherein the compound is Formula (I) or a pharmaceutically acceptable salt thereof and the flexible subunit of Formula (I) is a methylene subunit.

6. The method of claim 1, wherein the compound is Formula (I) or a pharmaceutically acceptable salt thereof and X in Formula (I) comprises (ix)

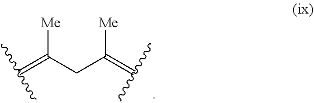 [SMILES: *=C(C)CC(=*)C]

7. The method of claim 1, wherein the compound is Formula (I) or a pharmaceutically acceptable salt thereof and each of the two or more conformational constraints in Formula (I) is independently selected from the group consisting of (x), (x′), (x″), and (x″′):

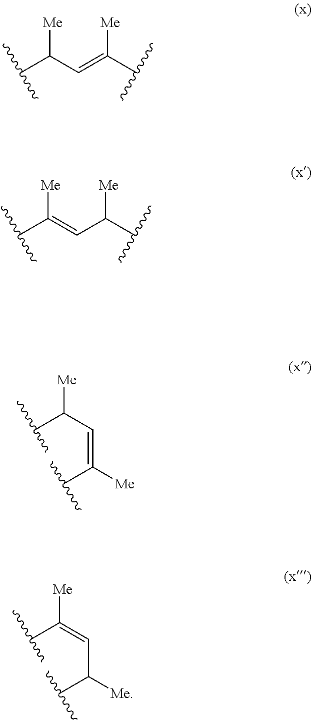 [SMILES: */C(C)=C/C(*)C ; */C(C)=C/C(*)C ; */C(C)=C\C(*)C ; */C(C)=C\C(*)C]

8. The method of claim 7, wherein the two or more conformational constraints are joined by a C₁₋₆-alkylene.

9. The method of claim 1, wherein the compound is Formula (I) or a pharmaceutically acceptable salt thereof and A comprises A²-R¹⁵—, wherein A² is a polar head group, and R¹⁵ is a bond, a C₁₋₆-alkyl, or a C₂-C₆ alkenyl.

10. The method of claim 1, wherein the compound has a structure of Formula (I-A): 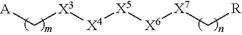
wherein X³ is a bond, an alkyl, or a conformational constraint;
X⁴ is a bond or a flexible subunit;
X⁵ is a conformational constraint;
X⁶ is a bond or a flexible subunit; and
X⁷ is a bond, an alkyl, or a conformational constraint.

11. The method of claim 10, wherein at least one of X³ or X⁷ is a conformational constraint.

12. The method of claim 1, wherein the compound is Formula (II) or a pharmaceutically acceptable salt thereof and each of the one or more acyclic conformational constraints in Formula (II) is (x), (x′), (x″), or (x″′): 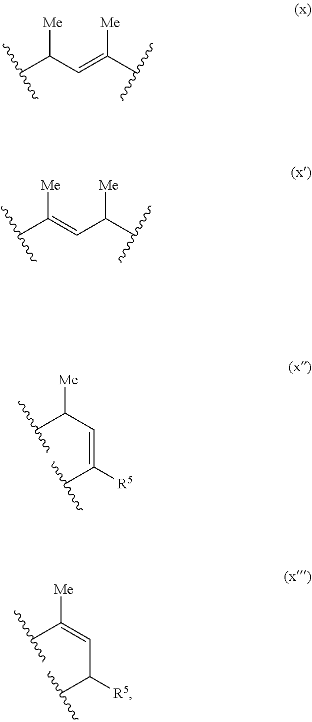
wherein R⁵ is hydrogen or C₁₋₆-alkyl.

13. The method of claim 12, wherein each acyclic conformational constraint in Formula (II) is joined by a C₁₋₆-alkylene.

14. The method of claim 1, wherein the compound is Formula (II) or a pharmaceutically acceptable salt thereof and X in Formula (II) comprises:

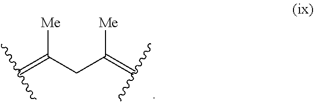 [SMILES: *=C(C)CC(=*)C]

15. The method of claim 1, wherein the compound is Formula (II) or a pharmaceutically acceptable salt thereof and X in Formula (II) is: 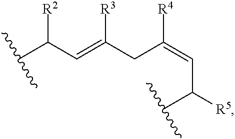
wherein:
R² is C₁₋₆-alkyl;
R³ is C₁₋₆-alkyl;
R⁴ is C₁₋₆-alkyl; and
R⁵ is hydrogen or C₁₋₆-alkyl.

16. The method of claim 1, wherein the compound is Formula (III) or a pharmaceutically acceptable salt thereof and
X¹ in Formula (III) is (x.1) or (x.2): 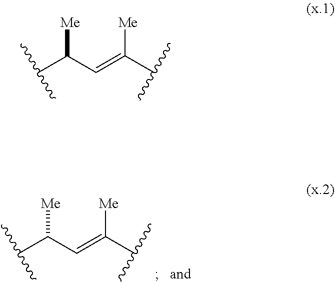 [SMILES: */C(C)=C/[C@@H](*)C ; */C(C)=C/[C@H](*)C]
X² in Formula (III) is (x″′.1) or (x″′.2):

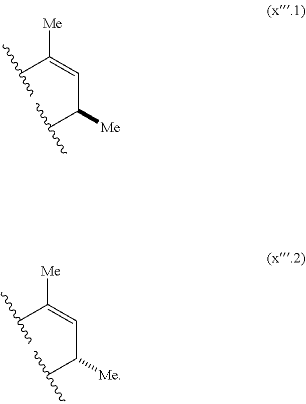 [SMILES: */C(C)=C\[C@H](*)C ; */C(C)=C\[C@@H](*)C]

17. The method of claim 16, wherein each of R², R³, and R⁴ are methyl or a partially or fully deuterated methyl, and R⁵ is hydrogen, deuterium, methyl, or a partially or fully deuterated methyl.

18. The method of claim 1, wherein the compound is selected from one of the following compounds:

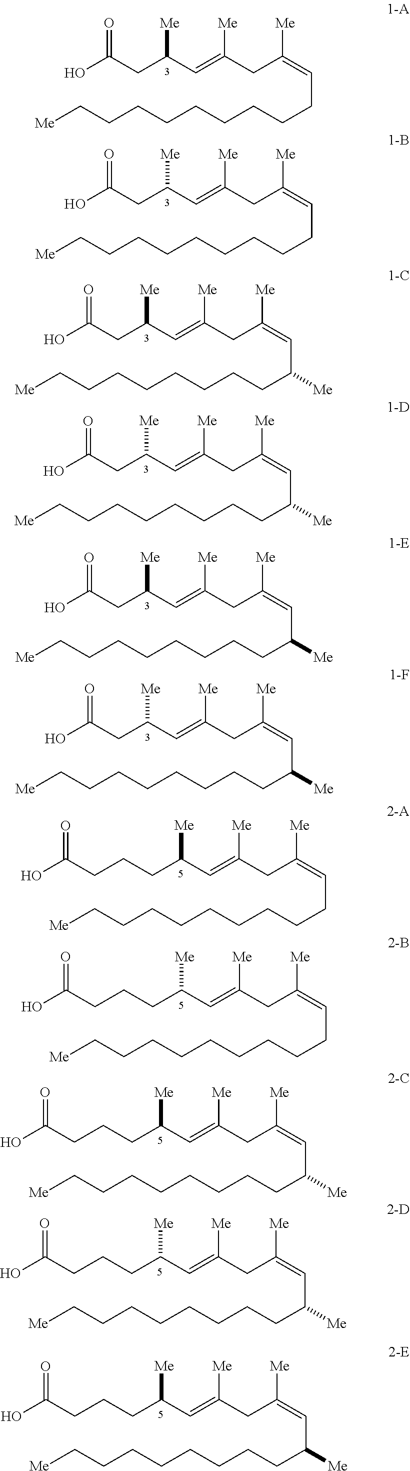

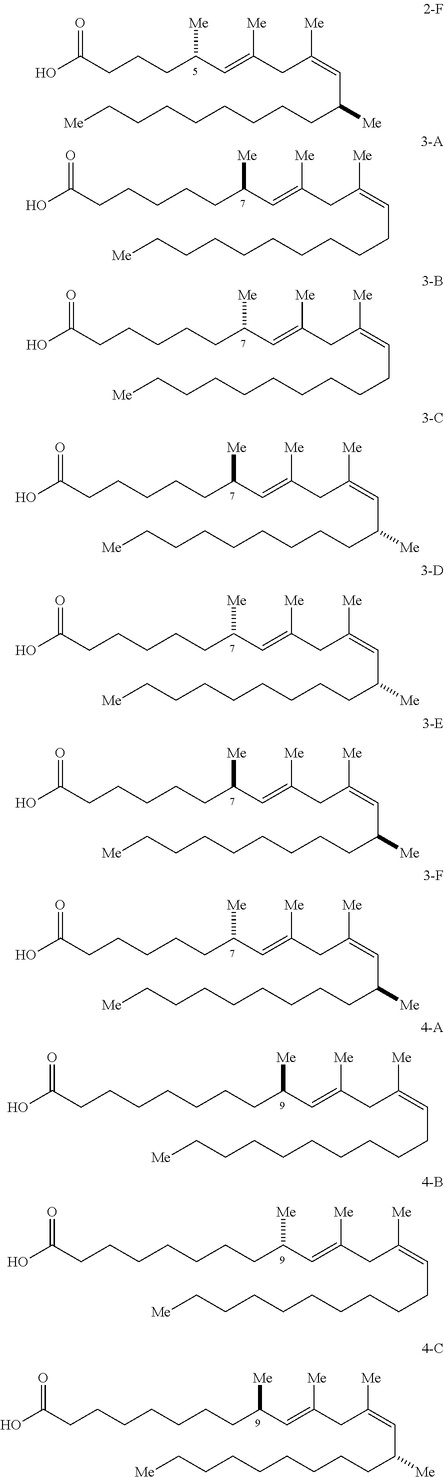

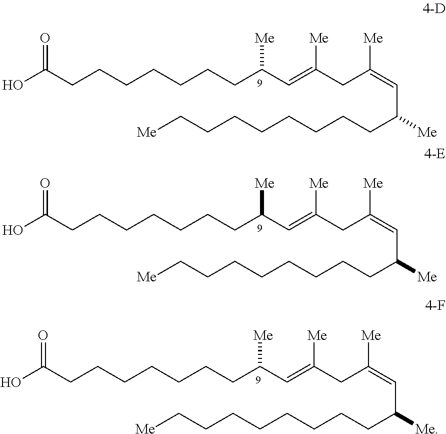 [SMILES: CCCCCCCCC[C@@H](C)/C=C(/C)C/C(C)=C/[C@@H](C)CCCCCCCC(=O)O ; CCCCCCCCC[C@H](C)/C=C(/C)C/C(C)=C/[C@H](C)CCCCCCCC(=O)O ; CCCCCCCCC[C@H](C)/C=C(/C)C/C(C)=C/[C@@H](C)CCCCCCCC(=O)O]

19. A method of conformational profiling of ligand binding to nuclear receptor TLX (NR2E1) comprising:
preparing a set of mimetics of the target fatty acid; and
assessing the functional activity of each member of the set of fatty acid mimetics at nuclear receptor TLX (NR2E1).

20. The method of claim 19, further comprising at least one of the following steps:
identifying one or more subpopulations of the set that have structural features that are preferred for modulating activity of nuclear receptor TLX (NR2E1);
preparing a subset of fatty acid mimetics comprising the structural features of the one or more subpopulations, wherein the mimetics of the subset comprise additional structural rigidification compared to the mimetics of the subpopulation; and/or
creating a model that shows conformation of the target fatty acid bound to nuclear receptor TLX (NR2E1), wherein the model is based on the assessment of which of the mimetics of the set bound to nuclear receptor TLX (NR2E1), wherein the model optionally comprises a lattice that depicts the three-dimensional space of the target fatty acid bound to nuclear receptor TLX (NR2E1).
